(19) United States
(12) Patent Application Publication
(10) Pub. No.: US 2024/0307335 A1
(43) Pub. Date: Sep. 19, 2024

(54) IRG Blockade to Armor CAR T Cells Against Myeloid Dysfunction

(71) Applicant: ROSWELL PARK CANCER INSTITUTE CORPORATION, Buffalo, NY (US)

(72) Inventor: Marco DAVILA, Buffalo, NY (US)

(21) Appl. No.: 18/605,289

(22) Filed: Mar. 14, 2024

Related U.S. Application Data
(60) Provisional application No. 63/452,019, filed on Mar. 14, 2023.

(51) Int. Cl. A61K 31/198; A61K 39/00

(52) U.S. Cl. CPC A61K 31/198; A61K 39/4611; A61K 39/4631; A61K 2239/26

(57) ABSTRACT

Disclosed herein are methods and compositions relating to the resistance of adoptive immunotherapy caused by the induction of iNOS in tumor associated macrophage. In one aspect, disclosed herein are methods of methods of treating, inhibiting, reducing, decreasing, ameliorating, and/or preventing a cancer and/or metastasis (such as, for example large B cell lymphoma) in a subject comprising administering to the subject an adoptive immune cell immunotherapy (including, but not limited to administration of chimeric antigen receptor (CAR) T cells, CAR natural killer (NK) cells, tumor infiltrating lymphocytes (TILs)) and an agent that blocks dysregulation of the citric acid cycle (such as, for example, an agent that inhibits inducible nitric oxide synthase (iNOS) and/or an agent that inhibits immune responsive gene 1 (IRG1)) or administering to the subject an adoptive immune cell immunotherapy (including, but not limited to administration of chimeric antigen receptor (CAR) T cells, CAR natural killer (NK) cells, tumor infiltrating lymphocytes (TILs)) wherein the immune cell has been modified to disrupt expression of IRG1.

### CROSS REFERENCE TO RELATED APPLICATIONS

This application claims the benefit of priority to U.S. Provisional Application No. 63/452,019, filed Mar. 14, 2023, which is incorporated by reference herein in its entirety.

### GOVERNMENT SUPPORT CLAUSE

This invention was made with government support under Grant Nos. R01HL167232 awarded by the National Institutes of Health. The Government has certain rights in the invention.

### BACKGROUND

The success of CD19-targeted CAR T cell therapies has advanced the treatment of B cell malignancies. However, a substantial proportion of patients with LBCL experience primary resistance or relapse, supporting the need to augment CAR T cell efficacy. Factors hindering the effectiveness of CAR T cell therapy include a high tumor burden prior to CAR T cell infusion, loss of or decreased CD19 expression on tumor cells, tumor genetic alterations, and the highly differentiated or dysfunctional state of CAR T cells. Recent studies have also emphasized the importance of the TME factors in determining clinical outcomes in CAR T therapy patients.

The TME of B cell lymphoma contains various immune cell types, including macrophages, myeloid-derived suppressor cells (MDSCs), and regulatory T cells (Tregs), which can impede the recruitment, expansion, and activity of T cells, including endogenous T cells and infused CAR T cells. In LBCL, the pre-infusion TME, characterized by elevated expression of genes associated with immune suppression and diminished T cell-related signatures, is correlated with relapse after CAR T cell therapy. In contrast, higher rates of complete response are associated with the TME that exhibits immune gene signatures linked to cytotoxic T cell activation and is enriched with chemokines and cytokines that potentiate T cell involvement.

Macrophages perform diverse biological functions in response to tissue pathophysiology and environmental cues. They are central players of immune-mediated toxicities associated with CAR T cell therapy, such as the cytokine release syndrome (CRS), which is in part mediated through the release of inflammatory cytokines by myeloid cells. Additionally, macrophages contribute to cancer progression in various cancers by suppressing T cell effector function through multiple processes, including the expression of inhibitory checkpoint ligands such as programmed cell death-ligand 1 (PD-L1), secretion of inhibitory cytokines such as TGF-βand IL-10, and depletion of amino acids, including arginine and tryptophan. What are needed are new compositions and therapeutic methods that avoid suppressive effects of macrophage.

### SUMMARY

Chimeric antigen receptor (CAR) T cell therapies have revolutionized B cell malignancy treatment, but subsets of patients with large B cell lymphoma (LBCL) experience primary resistance or relapse after CAR T cell treatment. CAR T cell-produced interferon-gamma (IFN-γ) can promote the expression of inducible nitric oxide synthase (iNOS, NOS2) in macrophages, impairing CAR T cell effector function. CAR T cell metabolism can be compromised by iNOS-dependent depletion of glycolytic intermediates and rewiring of the TCA cycle. Inhibition of iNOS can enhance the CAR T cell treatment efficacy in B cell tumor-bearing mice. The disclosed findings highlight the impact of targeting iNOS in tumor-associated macrophages (TAMs) to improve CAR T cell therapy outcomes in LBCL patients.

Disclosed herein are methods of treating, inhibiting, reducing, decreasing, ameliorating, and/or preventing a cancer and/or metastasis (such as, for example large B cell lymphoma) or an autoimmune disease in a subject comprising administering to the subject an adoptive immune cell immunotherapy (including, but not limited to administration of chimeric antigen receptor (CAR) T cells, CAR natural killer (NK) cells, tumor infiltrating lymphocytes (TILs)) and an agent that blocks dysregulation of the citric acid cycle (such as, for example, an agent that inhibits inducible nitric oxide synthase (iNOS) and/or an agent that inhibits immune responsive gene 1 (IRG1)). In one aspect, the immune cells in the adoptive immune cell immunotherapy are CD8+ T cells, CD4+ T cells, NK cells, or NK T cells.

In one aspect, disclosed herein are methods of treating, inhibiting, reducing, decreasing, ameliorating, and/or preventing a cancer and/or metastasis or an autoimmune disease of any preceding aspect, wherein the iNOS inhibitor and/or IRG1 inhibitor is an antibody, antibody fragment, small molecule (such as, for example, N⁶-(1-Iminoethyl)-lysine, hydrochloride (L-NIL), siRNA, or oligonucleotide that disrupts iNOS transcription, expression, or binding.

Also disclosed herein are methods of treating, inhibiting, reducing, decreasing, ameliorating, and/or preventing a cancer and/or metastasis (such as, for example large B cell lymphoma) or an autoimmune disease in a subject comprising administering to the subject an adoptive immune cell immunotherapy (including, but not limited to administration of chimeric antigen receptor (CAR) T cells, CAR natural killer (NK) cells, tumor infiltrating lymphocytes (TILs)) wherein the immune cell has been modified to disrupt expression of IRG1. In one aspect, the immune cells in the adoptive immune cell immunotherapy are CD8+ T cells, CD4+ T cells, NK cells, or NK T cells.

In one aspect, disclosed herein are methods of treating, inhibiting, reducing, decreasing, ameliorating, and/or preventing a cancer and/or metastasis or an autoimmune disease of any preceding aspect, wherein the expression of IRG1 is disrupted by a clustered regularly interspaced short palindromic repeat (CRISPR)/CRISPR-associated (Cas)integration systems (such as, for example a Class 2 CRISPR/Cas integration system, including by not limited to a CRISPR/Cas9 integration system) that targets iNOS or IRG1. In some aspects, the immune cell is modified ex vivo.

Also disclosed herein are modified chimeric antigen receptor (CAR) immune cells or tumor infiltrating lymphocytes (such as, for example, wherein immune cells or TILs are a CD8+ T cells, CD4+ T cells, NK cells, or NK T cells) comprising a disrupted IRG1 gene.

In one aspect, disclosed herein are methods of treating, inhibiting, reducing, decreasing, ameliorating, and/or preventing a cancer and/or metastasis (such as, for example large B cell lymphoma) or an autoimmune disease in a subject comprising administering to the subject comprising administering to the subject the modified CAR immune cell or modified TIL of any preceding aspect.

Also disclosed herein are methods of rescuing non-durable responses (NDR) to an adoptive immune cell immunotherapy for a cancer or an autoimmune disease in a subject comprising a) obtaining previously adoptively transferred immune cells from the subject or macrophage from tumor microenvironment (TME) of the subject, b) measuring the expression level of immune responsive gene 1 (IRG1) in the transferred immune cell or inducible nitric oxide synthase (iNOS) in the macrophage; and c) administering to the subject an agent that inhibits IRG1 and/or iNOS or administering to the subject chimeric antigen receptor (CAR) immune cell comprising a disrupted IRG1 gene when the expression level of IRG1 in the adoptively transferred immune cell has increased relative to a control or the expression level of iNOS in the macrophage from the TME has increased relative to a control.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIG. 1A, FIG. 1B, FIG. 1C, FIG. 1D, FIG. 1E, and FIG. 1F depict immune cell composition of pre-CAR T cell treatment TME. FIG. 1A and FIG. 1B show data for patient tumor biopsies taken prior to lymphodepletion conditioning therapy and axi-cel infusion in LBCL patients (DR, 18 patients; NDR, 26 patients). FIG. 1A shows a heatmap showing relative abundances of CIBERSORTx deconvoluted immune cell types. FIG. 1B shows gene set enrichment analysis of M2-like macrophage signatures. FIG. 1C, FIG. 1D, FIG. 1E, and FIG. 1F show multiplex immunofluorescence analysis of patient tumor tissue microarray obtained prior to lymphodepletion conditioning therapy and axi-cel infusion (DR, 9 cores from 6 patients; NDR, 12 cores from 5 patients). Proportion of CD3⁺, CD4⁺CD3⁺, CD4⁻CD3⁺, and Foxp3⁺CD4+ T cells in DAPI⁺ cells. Data in FIG. 1C, FIG. 1D, FIG. 1E, and FIG. 1F are the mean±SEM. Statistical significance was determined by unpaired two-tailed t tests with Welch's correction. ns, not significant; DR, durable response; NDR, non-durable response.

FIG. 2A, FIG. 2B, FIG. 2C, FIG. 2D, and FIG. 2E depict that macrophages in the pre-CAR T cell treatment TME are linked to therapeutic responses to CAR T cell therapy in LBCL patients. FIG. 2A shows bulk RNA-seq analysis on patient tumor biopsies taken before lymphodepletion conditioning therapy and axi-cel infusion (DR, 18 patients; NDR, 26 patients). Percentages of M0, M1, and M2-like macrophages based on CIBERSORTx. FIG. 2B shows progression-free survival in patients stratified according to the abundance of CIBERSORTx-defined M2-like macrophages (“Low” represents patients with a <5% M2 macrophage population, 14 patients; “High” represents patients with a >10% M2 macrophage population, 19 patients). FIG. 2C, FIG. 2D, and FIG. 2E show multiplex immunofluorescence analysis of patient tumor tissue microarray obtained prior to lymphodepletion conditioning therapy and axi-cel infusion (DR, 9 cores from 6 patients; NDR, 12 cores from 5 patients). FIG. 2C shows illustrative images of LBLC tumors showing PAX5 (Opal 690, Purple), CD4 (Opal 570, Yellow), CD3 (Opal 650, Green), Foxp3 (Opal 620, Magenta), CD163 (Opal 540, Orange), CD68 (Opal 520, Cyan), and DAPI (Blue). FIG. 2D and FIG. 2E show the proportion of intratumor CD68⁺ macrophages and CD163⁺CD68⁺M2-like macrophages in DAPI⁺ cells. Data in FIG. 2A, FIG. 2D, and FIG. 2E are the mean±SEM. Statistical significance was determined by unpaired two-tailed t tests with Welch's correction (FIG. 2A, FIG. 2D, and FIG. 2E) or log-rank Mantel-Cox test (FIG. 2B). *, P<0.05; **, P<0.01; ns, not significant; DR, durable response; NDR, non-durable response.

FIG. 3A, FIG. 3B, FIG. 3C, FIG. 3D, FIG. 3E, FIG. 3F, FIG. 3G, FIG. 3H, FIG. 3I, and FIG. 3J depict that exposure of imMac provokes CAR T cell dysfunction. FIG. 3A shows the schematic for FIG. 3B, FIG. 3C, FIG. 3D, FIG. 3E, and FIG. 3F where CAR T cells and Eμ-myc cells were cocultured with unMac or imMac or without macrophages. FIG. 3B shows CAR T cell death at 48 h assessed by 7-aminoactinomycin D (7-AAD) incorporation viaflow cytometry (n=4). FIG. 3C shows DNA replication of CAR T cells at 42 h measured by bromodeoxyuridine (BrdU) incorporation via flow cytometry (n=4). FIG. 3D shows CAR T cell expansion measured over 48 h by a live cell analysis system (n=4). FIG. 3E shows total CAR expression levels in CAR T cells at 48 h assessed via flow cytometry (n=4). FIG. 3F shows surface CAR expression levels in CAR T cells at 48 h detected by staining single-chain variable fragments (scFv) of CAR with protein L and analyzed via flow cytometry (n=4). FIG. 3G shows the schematic for FIG. 3H, FIG. 3I, and FIG. 3J where CAR T cells were isolated after initial coculture with Eμ-myc cells with unMac or imMac or without macrophages (No Mac) for 48 h, subsequently cocultured with fresh Eμ-myc cells. FIG. 3H and FIG. 3I show levels of IFN-γ and TNF-α in coculture supernatants of CAR T cells and Eμ-myc cells at 36 h analyzed by an automated enzyme-linked immunosorbent assay (ELISA) (n=4). FIG. 3J shows lysis of Eμ-myc cells by CAR T cells at 36 h assessed using a bioluminescence assay (n=4). All data are the mean±SD. Statistical significance was determined by one-way ANOVA with Bonferroni correction for multiple comparisons. *, P<0.05; ***, P<0.001; ****, P<0.0001; ##, P<0.01; ###, P<0.001; ####, P<0.0001; BM, bone marrow; MFI, mean fluorescence intensity; exp: exposed, isolated CAR T cells from prior coculture.

FIG. 4A and FIG. 4B depict that exposure of imMac suppresses CAR T cell expansion. FIG. 4A shows construct maps encoding anti-CD19 CAR. All included a 5′ long terminal repeat (LTR), anti-murine CD19 single-chain variable fragment (scFv), CD8 transmembrane and hinge domain (CD8 ™), glycine-serine linker (G/S), mCherry, and 3′ LTR. 19dz CAR is 1st generation CAR containing truncated CD3ξ chain (ΔCD3ξ). 1928z CAR is 2nd generation CAR containing CD28 costimulatory domain and CD3ξ chain. FIG. 4B shows representative IncuCyte images taken at 0, 24, and 40 h during cocultures of CAR T cells and Eμ-myc cells with unMac or imMac or without macrophages.

FIG. 5A, FIG. 5B, FIG. 5C, FIG. 5D, FIG. 5E, FIG. 5F, FIG. 5G, and FIG. 5H depict that CAR T cell-exposed imMac upregulates iNOS. FIG. 5A shows the schematic for FIG. 5B, FIG. 5C, FIG. 5D, FIG. 5E, and FIG. 5F where CAR T cells and Eμ-myc cells were cocultured with unMac or imMac or without macrophages (No Mac) for 24 h. Coculture supernatants were analyzed by global metabolomics using LC-MS. FIG. 5B and FIG. 5C shows quantification of relative abundance of metabolites in the supernatants derived from cocultures containing imMac versus No Mac (FIG. 5B) or unMac (FIG. 5C). FIG. 5D, FIG. 5E, and FIG. 5F show arginine, ornithine, and citrulline levels in the supernatants of coculture groups (n=3). The data were normalized to the mean value of CAR T and Eμ-myc cell cocultures without macrophages. FIG. 5G shows flow cytometry analysis of expression of ARG-1 and iNOS in unMac or imMac cocultured with Eμ-myc cells in the presence or absence of CAR T cells at 24 h (n=3). FIG. 5H shows NO levels in the supernatants derived from cocultures of CAR T cells and Eμ-myc cells with unMac or imMac or without macrophages at 48 h analyzed by Griess assay (n=4). Data in FIG. 5D, FIG. 5E, FIG. 5F, FIG. 5G, and FIG. 5H are presented in mean±SD. Statistical significance was determined by unpaired two-tailed Student's t tests (FIG. 5B, FIG. 5C, and FIG. 5G) or one-way ANOVA with Bonferroni correction for multiple comparisons (FIG. 5D, FIG. 5E, FIG. 5F, and FIG. 5H). ***, P<0.001; ****, P<0.0001; ns, not significant; FC, fold change; LC-MS, liquid chromatography-mass spectrometry; NO, nitric oxide.

FIG. 6A, FIG. 6B, and FIG. 6C depict that exposure to CAR T cells induces phenotypic changes in macrophages. FIG. 6A shows arginine metabolic pathways. FIG. 6B shows data for CAR T cells and Eμ-myc cells cocultured with unMac or imMac for 24 h. Expression of ARG-1 and iNOS in CD3+ T cells or CD19⁺ Eμ-myc cells were analyzed by flow cytometry. FIG. 6C shows flow cytometry analysis of expression of PD-L1 in unMac or imMac cocultured with Eμ-myc cells in the presence or absence of CAR T cells at 24 h (n=3). Data in FIG. 6C are presented in mean±SD. Statistical significance was determined by unpaired two-tailed Student's t tests. ****, P<0.0001. ARG-1, arginase-1; iNOS, inducible nitric oxide synthase; NO, nitric oxide.

FIG. 7A, FIG. 7B, FIG. 7C, FIG. 7D, FIG. 7E, FIG. 7F, FIG. 7G, FIG. 7H, FIG. 7I, FIG. 7J, FIG. 7K, and FIG. 7L depict that arginine metabolism by imMac mediates impairment of CAR T cell function. FIG. 7A shows the arginine metabolic pathway and the inhibitors of respective enzymes. FIG. 7B and FIG. 7C CAR T cells and Eμ-myc cells were cocultured with unMac or imMac or without macrophages in the culture media containing supraphysiological (1.15 mM) (FIG. 7B) or physiological (150 PM) (FIG. 7C) concentration of arginine in the presence or absence of nor-NOHA (50 PM). CAR T cell expansion was measured over 48 h by a live cell analysis system (n=4). FIG. 7D shows flow cytometry analysis of expression of ARG-1 and PD-L1 in WT or iNOS^{-/-} imMac cocultured with CAR T cells and Eμ-myc cells at 24 h. FIG. 7E shows analysis of NO levels in coculture supernatants of CAR T cells and Eμ-myc cells with or without WT or iNOS^{-/-} unMac or imMac in the presence or absence of L-NIL (50 μM) at 48 h using Griess assay (n=4). FIG. 7F shows analysis of NO levels in coculture supernatants of CAR T cells and Eμ-myc cells with or without WT or iNOS^{-/-} unMac or imMac at 48 h using Griess assay (n=4). FIG. 7G, FIG. 7H, and FIG. 7I show citrulline, arginine, and ornithine levels in coculture supernatants of CAR T cells and Eμ-myc cells with or without WT or iNOS^{-/-} unMac or imMac in the presence or absence of L-NIL (50 PM) (n=3). Graphs contain control data also used in FIG. 5D, FIG. 5E, and FIG. 5F. FIG. 7J shows data for CAR T cells and Eμ-myc cells cocultured in the increasing concentrations of citrulline. CAR T cell expansion was measured over 48 h by a live cell analysis system (n=4). FIG. 7K and FIG. 7L show data for isolated CAR T cells were cocultured with fresh Eμ-myc cells after initial coculture of CAR T cells and Eμ-myc cells in the presence of NCX-4016 (100 PM) or PNT (50 μM) or 0.01% DMSO (Veh) for 48 h. Levels of IFN-γ and TNF-α in coculture supernatants at 24 h were measured by ELISA (n=4). All data are presented in mean±SD. Data in FIG. 7E, FIG. 7F, FIG. 7G, FIG. 7H, FIG. 7I, FIG. 7J, FIG. 7K, and FIG. 7L, statistical significance was determined by one-way ANOVA with Bonferroni correction for multiple comparisons. **, P<0.01; ****, P<0.0001; ns, not significant; NO, nitric oxide; PNT, peroxynitrite; exposed, isolated CAR T cells from prior coculture.

FIG. 8A, FIG. 8B, FIG. 8C, FIG. 8D, FIG. 8E, FIG. 8F, FIG. 8G, FIG. 8H, FIG. 8I, FIG. 8J depict that iNOS upregulation in imMac drives suppression of CAR T cell function. FIG. 8A shows data for CAR T cells and Eμ-myc cells cocultured with unMac or imMac or without macrophages in the presence or absence of L-NIL (50 μM). CAR T cell expansion was measured over 48 h by a live cell analysis system (n=4). FIG. 8B, FIG. 8C, and FIG. 8D show data for isolated CAR T cells cocultured with fresh Eμ-myc cells after initial coculture of CAR T cells and Eμ-myc cells with unMac or imMac or without macrophages (No Mac) in the presence or absence of L-NIL (50 PM) for 48 h. FIG. 8B shows luciferase-based lysis of Eμ-myc cells by CAR T cells assessed at 24 h (n=4). FIG. 8C and FIG. 8D show levels of IFN-γ and TNF-α in coculture supernatants at 36 h analyzed by ELISA (n=4). FIG. 8E shows data for CAR T cells and Eμ-myc cells cocultured with WT or iNOS^{-/-} unMac or imMac or without macrophages. CAR T cell expansion was measured over 48 h by a live cell analysis system (n=4). FIG. 8F shows data for isolated CAR T cells cocultured with fresh Eμ-myc cells after initial coculture of CAR T cells and Eμ-myc cells with WT or iNOS^{-/-} unMac or imMac or without macrophages (No Mac) for 48 h. Luciferase-based lysis of Eμ-myc cells by CAR T cells was assessed at 36 h (n=4). FIG. 8G shows data for CAR T cells cocultured with Eμ-myc cells in the presence of NCX-4016 or 0.01% DMSO (Veh). CAR T cell expansion was measured over 48 h by a live cell analysis system (n=3-4). FIG. 8H shows data for CAR T cells cocultured with Eμ-myc cells in the presence or absence of PNT. CAR T cell expansion was measured over 48 h by a live cell analysis system (n=4). FIG. 8I shows data for isolated CAR T cells cocultured with fresh Eμ-myc cells after initial coculture of CAR T cells and Eμ-myc cells in the presence of NCX-4016 (100 μM) or PNT (50 μM) or 0.01% DMSO (Veh) for 48 h. Luciferase-based lysis of Eμ-myc cells by CAR T cells was assessed at 24 h (n=4). FIG. 8J shows data for CAR T cells and Eμ-myc cells cocultured with or without imMac in the presence or absence of c-PTIO. CAR T cell expansion was measured over 48 h by a live cell analysis system (n=3). All data are presented in mean±SD. Statistical significance was determined by unpaired two-tailed Student's t tests (FIG. 8A, FIG. 8B, FIG. 8E, FIG. 8F, FIG. 8G, FIG. 8H, FIG. 8I, and FIG. 8J) or one-way ANOVA with Bonferroni correction for multiple comparisons (FIG. 8C and FIG. 8D). *, P<0.05; **, P<0.01; ***, P<0.001; ****, P<0.0001; ####, P<0.0001; PNT, peroxynitrite; c-PTIO, carboxyl-PTIO; exp: exposed, isolated CAR T cells from prior coculture.

FIG. 9A, FIG. 9B, FIG. 9C, FIG. 9D, FIG. 9E, and FIG. 9F depict that CAR T cell-derived IFN-γ induces iNOS expression in imMac. FIG. 9A, FIG. 9B, and FIG. 9C show data for CAR T cells and Eμ-myc cells cocultured with unMac or imMac in the presence of anti-IFN-γ (10 μg/mL) or IsoCon (10 μg/mL). FIG. 9A shows expression of ARG-1 and iNOS in unMac or imMac analyzed at 24 h via flow cytometry (n=3). FIG. 9B shows NO levels in coculture supernatants at 44 h analyzed by Griess assay (n=4). FIG. 9C shows CAR T cell expansion measured over 48 h by a live cell analysis system (n=4). FIG. 9D, FIG. 9E, and FIG. 9F show data for isolated CAR T cells cocultured with fresh Eμ-myc cells after initial coculture of CAR T cells and Eμ-myc cells with unMac or imMac in the presence of anti-IFN-γ (10 μg/mL) or IsoCon (10 μg/mL) for 48 h. FIG. 9D shows luciferase-based lysis of Eμ-myc cells by CAR T cells assessed at 24 h (n=4). FIG. 9E and FIG. 9F show levels of IFN-γ and TNF-α in the coculture supernatants at 24 h analyzed by ELISA (n=4). All data are presented in mean±SD. Statistical significance was determined by unpaired two-tailed Student's t tests (FIG. 9A, FIG. 9C, and FIG. 9D) or one-way ANOVA with Bonferroni correction for multiple comparisons (FIG. 9B, FIG. 9E, and FIG. 9F). *, P<0.05; **, P<0.01; 33***, P<0.001; ****, P<0.0001; IsoCon, isotype control; NO, nitric oxide; exp: exposed, isolated CAR T cells from prior coculture.

FIG. 10A, FIG. 10B, and FIG. 10C depict that IFN-γ^{-/-} CAR T cells do not trigger iNOS in macrophages. WT or IFN-γ^{-/-} CAR T cells were cocultured with Eμ-myc cells with unMac or imMac. FIG. 10A shows expression of ARG-1 and iNOS in unMac or imMac at 24 h analyzed by flow cytometry (n=3). FIG. 10B shows NO levels in coculture supernatants at 48 h analyzed by Griess assay (n=4). FIG. 10C shows CAR T cell expansion measured over 48 h by a live cell analysis system (n=4). All data are presented in mean±SD. Statistical significance was determined by unpaired two-tailed Student's t tests (FIG. 10A and FIG. 10C) or one-way ANOVA with Bonferroni correction for multiple comparisons (FIG. 10B). ***, P<0.001;****, P<0.0001; WT, wildtype; NO, nitric oxide.

FIG. 11A, FIG. 11B, FIG. 11C, FIG. 11D, FIG. 11E, FIG. 11F, FIG. 11G, FIG. 11H, FIG. 11I, FIG. 11J, FIG. 11K, FIG. 11L, and FIG. 11M depict that iNOS-expressing imMac induces CAR T cell metabolic dysregulation. FIG. 11A, FIG. 11B, FIG. 11C, FIG. 11D, FIG. 11E, FIG. 11F, FIG. 11G, FIG. 11H, FIG. 11I, FIG. 11J, and FIG. 11K show data for CAR T cells and Eμ-myc cells cocultured with unMac or imMac or without macrophages (No Mac) in the presence or absence of L-NIL (50 PM). After coculture for 48 h, global metabolomics was performed on CAR T cells. FIG. 11A and FIG. 11B show quantification of relative abundance of metabolites in CAR T cells derived from cocultures with imMac versus No Mac (FIG. 11A) or unMac (FIG. 11B). FIG. 11C shows a schematic depicting altered metabolites associated with glycolysis pathway and TCA cycle. FIG. 11D, FIG. 11E, FIG. 11F, FIG. 11G, FIG. 11H, FIG. 11I, FIG. 11J, and FIG. 11K show levels of F1,6BP, G3P, DHAP, citrate, aconitate, succinate, malate, and itaconate (n=4). The data were normalized to the mean value of CAR T cells derived from cocultures without macrophages and L-NIL treatment. FIG. 11L shows data for CAR T cells cocultured with Eμ-myc cells in the presence or absence of 4-OI. CAR T cell expansion was measured over 48 h by a live cell analysis system (n=3-4). FIG. 11M shows data for CAR T cells and Eμ-myc cells cocultured with imMac or without macrophages (No Mac) in the presence or absence of L-NIL (50 μM). After coculture for 48 h, seahorse assay was performed on CAR T cells (n=6). ECAR was measured in response to glucose (Glc), ATP synthase inhibitor (Oligo), or hexokinase II inhibitor (2-DG). OCR was measured in response to Oligo, mitochondrial oxidative phosphorylation uncoupler (FCCP), or electron transport chain complex I/III inhibitor (Rot/AA). Data in FIG. 11D, FIG. 11E, FIG. 11F, FIG. 11G, FIG. 11H, FIG. 11I, FIG. 11J, and FIG. 11K are presented in mean±SD. Statistical significance was determined by unpaired two-tailed Student's t tests (FIG. 11A, FIG. 11B, and FIG. 11L) or one-way ANOVA with Bonferroni correction for multiple comparisons (FIG. 11D, FIG. 11E, FIG. 11F, FIG. 11G, FIG. 11H, FIG. 11I, FIG. 11J, and FIG. 11K). *, P<0.05; **, P<0.01; ***, P<0.001; ****, P<0.0001; ns, not significant; F1,6BP, 1,6-bisphosphate; G3P, glyceraldehyde 3-phosphate; DHAP, dihydroxyacetone phosphate; 4-OI, 4-octyl itaconate; ECAR, extracellular acidification rate; OCR, oxygen consumption rate; Oligo, oligomycin A; 2-DG, 2-deoxy-D-glucose; FCCP, carbonyl cyanide p-trifluoromethoxyphenylhydrazone; Rot/AA, rotenone, antimycin A; exp: exposed, isolated CAR T cells from prior coculture.

FIG. 12A, FIG. 12B, FIG. 12C, FIG. 12D, FIG. 12E, FIG. 12F, FIG. 12G, and FIG. 12H depict that itaconate is endogenously produced in imMac-exposed CAR T cells. CAR T cells and Eμ-myc cells were cocultured with unMac or imMac or without macrophages in the presence or absence of L-NIL (50 PM) for 48 h. FIG. 12A, FIG. 12B, FIG. 12C, FIG. 12D, FIG. 12E, FIG. 12F, and FIG. 12G show ¹³C₆-glucose tracing performed on isolated CAR T cells. FIG. 12A is a schematic showing ¹³C-labeled TCA cycle-related metabolites generated from ¹³C6-glucose. Colored circles represent ¹³C-labeled carbons. FIG. 12B, FIG. 12C, FIG. 12D, FIG. 12E, FIG. 12F, and FIG. 12G show levels of ¹³C-labeled citrate, aconitate, itaconate, α-ketoglutarate, fumarate, and malate (n=3). FIG. 12H shows immunoblotting performed on isolated CAR T cells. The 3-actin was used as a loading control. Data in FIG. 12B, FIG. 12C, FIG. 12D, FIG. 12E, FIG. 12F, and FIG. 12G are presented in mean±SD. Statistical significance was determined by one-way ANOVA with Bonferroni correction for multiple comparisons. *, P<0.05; **, P<0.01; ***, P<0.001; ****, P<0.0001; IDH, isocitrate dehydrogenase; IRG1, immune response gene 1; exp: exposed, isolated CAR T cells from prior coculture.

FIG. 13A, FIG. 13B, FIG. 13C, FIG. 13D, FIG. 13E, FIG. 13F, FIG. 13G, and FIG. 13H depict that iNOS inhibition improves efficacy of CAR T cell therapy. FIG. 13A shows experimental settings for FIG. 13B, FIG. 13C, FIG. 13D, and FIG. 13E where Eμ-myc cells were intraperitoneally injected into Rag1^{-/-} mice. Seven days later, WT 1928z, IFN-γ−/− 1928z, or WT 19dz CAR T cells were transferred into mice. Peritoneal lavage cells were obtained 24-40 h after CAR T cell transfer. FIG. 13B, FIG. 13C, and FIG. 13D show flow cytometry analysis of iNOS⁺ cells among CD11b⁺F4/80⁺ macrophages (FIG. 13B), ARG-1⁺ cells among CD11b⁺F4/80⁺ macrophages (FIG. 13C), and CD11b⁺F4/80⁺ macrophages among CD45⁺ cells (FIG. 13D) at 24 h after CAR T cell transfer (n=6/group). FIG. 13E shows data for CD11b⁺ peritoneal myeloid cells obtained 40 h following WT 1928z CAR T cell transfer (n=5 mice). CD11b⁺ cells were then ex vivo cocultured with fresh antigen-naïve CAR T cells and Eμ-myc cells in the presence or absence of L-NIL (50 μM) (coculture ratio, 1:1:1=CD11b⁺ cell:CAR T:Eμ-myc cell). Expansion of CAR T cells was measured over 48 h by a live cell analysis system (n=4-5). FIG. 13F shows experimental settings for FIG. 13G. FIG. 13G shows percentage of survival of tumor-bearing mice treated with WT 19dz or WT 1928z CAR T cells receiving L-NIL or PBS (vehicle). Results are from two pooled independent experiments (PBS, n=12 mice; L-NIL, n=12 mice; 19dz CAR T+PBS, n=11 mice; 19dz CAR T+L-NIL, n=12 mice; 1928z+PBS, n=28 mice; 1928z+L-NIL, n=34 mice). FIG. 13H shows multiplex immunofluorescence analysis of patient tumor tissue microarray obtained prior to lymphodepletion conditioning therapy and axi-cel infusion (DR, 9 cores from 6 patients; NDR, 12 cores from 5 patients). Proportion of iNOS⁺CD14⁺ cells in DAPI⁺ cells. Data in FIG. 13B, FIG. 13C, and FIG. 13D are presented in mean±SEM and data in FIG. 13E are presented in mean±SD. Statistical significance was determined by one-way ANOVA with Bonferroni correction for multiple comparisons (FIG. 13B, FIG. 13C, and FIG. 13D), unpaired two-tailed Student's t tests (FIG. 13E), log-rank Mantel-Cox test (FIG. 13G), or unpaired two-tailed t tests with Welch's correction (FIG. 13H). *, P<0.05; **, P<0.01; ***, P<0.001.

FIG. 14A and FIG. 14B depict that iNOS⁺ myeloid cells in the TME are associated with poor therapeutic responses to CAR T cell therapy. FIG. 14A shows a flow cytometry gating strategy for the identification of TAM expression of ARG-1 and iNOS in Eμ-myc cell tumors. FIG. 14B shows illustrative images of LBLC tumors showing CD14 (Opal 570, Orange), iNOS (Opal 620, Yellow), and DAPI (Blue).

FIG. 15 shows that iNOS in imMac disrupts TCA cycle progression in CAR T cells. Treatment of itaconate or 401 (cell-permeable itaconate) reduced CAR T cell expansion, while supplementation of DMKG (cell-permeable α-ketoglutarate) failed to rescue CAR T cell expansion. Endogenous itaconate production and accumulation may be driving CAR T failure.

FIG. 16 shows that CAR T cell-derived IFN-γ spurs upregulation of iNOS in imMac.

FIG. 17 shows that iNOS inhibition improves CAR T cell-mediated tumor control and survival.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the terms “optional” or “optionally” means that the subsequently described event or circumstance can or cannot occur, and that the description includes instances where said event or circumstance occurs and instances where it does not.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Compositions

In one aspect disclosed herein are modified chimeric antigen receptor (CAR) immune cells or tumor infiltrating lymphocytes (TILs) (such as, for example, immune cells or TILs are a CD8+ T cells, CD4+ T cells, NK cells, or NK T cells) comprising a disrupted IRG1 gene. It is understood and herein contemplated that the disclosed modified CAR immune cells or TILs can be to treat, inhibit, reduce, decrease, ameliorate, and/or prevent any disease where CAR immune cells or TILs would be employed as an immunotherapy. Thus, in one aspect, disclosed herein are methods of treating, inhibiting, reducing, decreasing, ameliorating, and/or preventing a cancer and/or metastasis (such as, for example large B cell lymphoma) or an autoimmune disease in a subject comprising administering to the subject comprising administering to the subject the CAR immune cell of any preceding aspect.

In some embodiments, the engineered cell (e.g., T cell, NK cell, NKT cell, B cell) comprising the chimeric antigen receptor (CAR) is created by a) obtaining a ribonucleoprotein (RNP) complex comprising a class 2 CRISPR/Cas endonuclease (Cas9) complexed with a corresponding CRISPR/Cas guide RNA and an AAV vector comprising a plasmid, nucleic acid, and/or construct comprising a polynucleotide sequence encoding a chimeric antigen receptor (CAR) polypeptide b) introducing the polynucleotide sequence encoding the CAR polypeptide and the RNP complex into a cell; wherein the polynucleotide sequence encoding the CAR polypeptide is introduced into the cell via infection with the AAV into the cell; wherein the RNP complex hybridizes to a target sequence within the genomic DNA of the cell and the cell's DNA repair enzymes insert the polynucleotide sequence encoding the CAR polypeptide into the host genome at the target sequence within the genomic DNA of the cell thereby creating the engineered cell. In some embodiments, the engineered cells are generated by the methods disclosed in International Application Nos. WO2022/093863 and WO2020/198675, which are incorporated herein by reference in their entireties.

In some embodiments, the plasmid, nucleic acid, or construct further comprises a murine leukemia virus derived (MND) promoter. In some embodiments, the serotype of the AAV comprises AAV6. In some embodiments, the vector is a single stranded AAV (ssAAV) or a self-complimentary AAV (scAAV).

### Methods of Use

The success of CD19-targeted CAR T cell therapies has advanced the treatment of B cell malignancies. However, a substantial proportion of patients with LBCL experience primary resistance or relapse, supporting the need to augment CAR T cell efficacy. Factors hindering the effectiveness of CAR T cell therapy include a high tumor burden prior to CAR T cell infusion, loss of or decreased CD19 expression on tumor cells, tumor genetic alterations, and the highly differentiated or dysfunctional state of CAR T cells. Recent studies have also emphasized the importance of the TME factors in determining clinical outcomes in CAR T therapy patients.

The TME of B cell lymphoma contains various immune cell types, including macrophages, myeloid-derived suppressor cells (MDSCs), and regulatory T cells (Tregs), which can impede the recruitment, expansion, and activity of T cells, including endogenous T cells and infused CAR T cells. In LBCL, the pre-infusion TME, characterized by elevated expression of genes associated with immune suppression and diminished T cell-related signatures, is correlated with relapse after CAR T cell therapy. In contrast, higher rates of complete response are associated with the TME that exhibits immune gene signatures linked to cytotoxic T cell activation and is enriched with chemokines and cytokines that potentiate T cell involvement.

Macrophages perform diverse biological functions in response to tissue pathophysiology and environmental cues. They are central players of immune-mediated toxicities associated with CAR T cell therapy, such as the cytokine release syndrome (CRS), which is in part mediated through the release of inflammatory cytokines by myeloid cells. Additionally, macrophages contribute to cancer progression in various cancers by suppressing T cell effector function through multiple processes, including the expression of inhibitory checkpoint ligands such as programmed cell death-ligand 1 (PD-L1), secretion of inhibitory cytokines such as TGF-β and IL-10, and depletion of amino acids, including arginine and tryptophan. However, prior to the present disclosure the crosstalk between adoptive T cell immunotherapy (including, but not limited to CAR T cells) and macrophage as a mechanism of therapeutic resistance was poorly defined.

Dysfunctional T cells in tumors exhibit diminished T cell receptor signaling, antitumor effector activity, and proliferation. Accumulating evidence indicates that metabolic deficiencies underlie T cell dysfunction in cancer. Tumor-infiltrating T cells display compromised glycolytic and oxidative metabolism with loss of mitochondrial mass and membrane potential. These findings suggest that the TME may present metabolic challenges to CAR T cells. However, the potential role of macrophages in contributing to the CAR T cell metabolic dysfunction remains unknown.

The disclosed compositions and methods can be used to treat any disease where uncontrolled cellular proliferation occurs such as cancers. A representative but non-limiting list of cancers that the disclosed compositions can be used to treat is the following: lymphomas such as B cell lymphoma (including, but not limited to large B cell lymphoma) and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers. Accordingly, disclosed herein are methods of treating, inhibiting, reducing, decreasing, ameliorating, and/or preventing a cancer and/or metastasis (such as, for example large B cell lymphoma) in a subject comprising administering to the subject an adoptive immune cell immunotherapy (including, but not limited to administration of chimeric antigen receptor (CAR) T cells, CAR natural killer (NK) cells, tumor infiltrating lymphocytes (TILs)) and an agent that blocks dysregulation of the citric acid cycle (such as, for example, an agent that inhibits inducible nitric oxide synthase (iNOS) and/or an agent that inhibits immune responsive gene 1 (IRG1)). In one aspect, the immune cells in the adoptive immune cell immunotherapy are CD8+ T cells, CD4+ T cells, NK cells, or NK T cells.

In one aspect, disclosed herein are methods of treating, inhibiting, reducing, decreasing, ameliorating, and/or preventing a cancer and/or metastasis of any preceding aspect, wherein the iNOS inhibitor and/or IRG1 inhibitor is an antibody, antibody fragment, small molecule (such as, for example, N-(1-Iminoethyl)-lysine, hydrochloride (L-NIL), siRNA, or oligonucleotide that disrupts iNOS transcription, expression, or binding.

Also disclosed herein are methods of treating, inhibiting, reducing, decreasing, ameliorating, and/or preventing a cancer and/or metastasis (such as, for example large B cell lymphoma) in a subject comprising administering to the subject an adoptive immune cell immunotherapy (including, but not limited to administration of chimeric antigen receptor (CAR) T cells, CAR natural killer (NK) cells, tumor infiltrating lymphocytes (TILs)) wherein the immune cell has been modified to disrupt expression of IRG1. In one aspect, the immune cells in the adoptive immune cell immunotherapy are CD8+ T cells, CD4+ T cells, NK cells, or NK T cells.

In one aspect, disclosed herein are methods of treating, inhibiting, reducing, decreasing, ameliorating, and/or preventing a cancer and/or metastasis, wherein the expression of iNOS and/or IRG1 is disrupted by a clustered regularly interspaced short palindromic repeat (CRISPR)/CRISPR-associated (Cas)integration systems (such as, for example a Class 2 CRISPR/Cas integration system, including by not limited to a CRISPR/Cas9 integration system) that targets iNOS or IRG1. In some aspects, the immune cell is modified ex vivo. In general, “CRISPR system” or “CRISPR integration system” refers collectively to transcripts and other elements involved in the expression of or directing the activity of CRISPR-associated “Cas” genes. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

It is understood and herein contemplated that the disclosed treatment regimens can used alone or in combination with any anti-cancer therapy known in the art including, but not limited to Abemaciclib, Abiraterone Acetate, Abitrexate® (Methotrexate), ABRAXANE® (Paclitaxel Albumin-stabilized Nanoparticle Formulation), ABVD, ABVE, ABVE-PC, AC, AC-T, ADCETRIS® (Brentuximab Vedotin), ADE, Ado-Trastuzumab Emtansine, Adriamycin® (Doxorubicin Hydrochloride), Afatinib Dimaleate, Afinitor® (Everolimus), Akynzeo® (Netupitant and Palonosetron Hydrochloride), ALDARA® (Imiquimod), Aldesleukin, ALECENSA® (Alectinib), Alectinib, Alemtuzumab, ALIMTA® (Pemetrexed Disodium), ALIQOPA® (Copanlisib Hydrochloride), ALKERAN™ for Injection (Melphalan Hydrochloride), ALKERAN™ Tablets (Melphalan), Aloxi® (Palonosetron Hydrochloride), Alunbrig® (Brigatinib), Ambochlorin® (Chlorambucil), Amboclorin® (Chlorambucil), Amifostine, Aminolevulinic Acid, Anastrozole, Aprepitant, Aredia® (Pamidronate Disodium), Arimidex® (Anastrozole), Aromasin® (Exemestane), Arranon® (Nelarabine), Arsenic Trioxide, Arzerra® (Ofatumumab), Asparaginase Erwinia chrysanthemi, Atezolizumab, Avastin® (Bevacizumab), Avelumab, Axitinib, Azacitidine, Bavencio® (Avelumab), BEACOPP, Becenum® (Carmustine), Beleodaq® (Belinostat), Belinostat, Bendamustine Hydrochloride, BEP, Besponsa® (Inotuzumab Ozogamicin), Bevacizumab, Bexarotene, Bexxar® (Tositumomab and Iodine I 131 Tositumomab), Bicalutamide, BiCNU® (Carmustine), Bleomycin, Blinatumomab, Blincyto® (Blinatumomab), Bortezomib, Bosulif® (Bosutinib), Bosutinib, Brentuximab Vedotin, Brigatinib, BuMel, Busulfan, Busulfex® (Busulfan), Cabazitaxel, Cabometyx® (Cabozantinib-S-Malate), Cabozantinib-S-Malate, CAF, Campath® (Alemtuzumab), Camptosar® (Irinotecan Hydrochloride), Capecitabine, CAPOX, Carac® (Fluorouracil—Topical), Carboplatin, CARBOPLATIN-TAXOL, Carfilzomib, Carmubris® (Carmustine), Carmustine, Carmustine Implant, Casodex® (Bicalutamide), CEM, Ceritinib, Cerubidine® (Daunorubicin Hydrochloride), Cervarix® (Recombinant HPV Bivalent Vaccine), Cetuximab, CEV, Chlorambucil, CHLORAMBUCIL-PREDNISONE, CHOP, Cisplatin, Cladribine, Clafen® (Cyclophosphamide), Clofarabine, Clofarex® (Clofarabine), Clolar® (Clofarabine), CMF, Cobimetinib, Cometriq® (Cabozantinib-S-Malate), Copanlisib Hydrochloride, COPDAC, COPP, COPP-ABV, Cosmegen® (Dactinomycin), Cotellic® (Cobimetinib), Crizotinib, CVP, Cyclophosphamide, Cyfos® (Ifosfamide), Cyramza® (Ramucirumab), Cytarabine, Cytarabine Liposome, Cytosar-U® (Cytarabine), Cytoxan® (Cyclophosphamide), Dabrafenib, Dacarbazine, Dacogen® (Decitabine), Dactinomycin, Daratumumab, Darzalex® (Daratumumab), Dasatinib, Daunorubicin Hydrochloride, Daunorubicin Hydrochloride and Cytarabine Liposome, Decitabine, Defibrotide Sodium, Defitelio® (Defibrotide Sodium), Degarelix, Denileukin Diftitox, Denosumab, DepoCyt® (Cytarabine Liposome), Dexamethasone, Dexrazoxane Hydrochloride, Dinutuximab, Docetaxel, Doxil® (Doxorubicin Hydrochloride Liposome), Doxorubicin Hydrochloride, Doxorubicin Hydrochloride Liposome, Dox-SL® (Doxorubicin Hydrochloride Liposome), DTIC-Dome® (Dacarbazine), Durvalumab, Efudex® (Fluorouracil—Topical), Elitek® (Rasburicase), Ellence® (Epirubicin Hydrochloride), Elotuzumab, Eloxatin® (Oxaliplatin), Eltrombopag Olamine, Emend® (Aprepitant), Empliciti® (Elotuzumab), Enasidenib Mesylate, Enzalutamide, Epirubicin Hydrochloride, EPOCH, Erbitux® (Cetuximab), Eribulin Mesylate, Erivedge® (Vismodegib), Erlotinib Hydrochloride, Erwinaze® (Asparaginase Erwinia chrysanthemi), Ethyol® (Amifostine), Etopophos Etopophos® (Etoposide Phosphate), Etoposide, Etoposide Phosphate, Evacet® (Doxorubicin Hydrochloride Liposome), Everolimus, Evista® (Raloxifene Hydrochloride), Evomela® (Melphalan Hydrochloride), Exemestane, 5-FU® (Fluorouracil Injection),5-FU® (Fluorouracil—Topical), Fareston® (Toremifene), Farydak® (Panobinostat), Faslodex® (Fulvestrant), FEC, Femara® (Letrozole), Filgrastim, Fludara® (Fludarabine Phosphate), Fludarabine Phosphate, Fluoroplex® (Fluorouracil—Topical), Fluorouracil Injection, Fluorouracil—Topical, Flutamide, Folex® (Methotrexate), Folex PFS® (Methotrexate), FOLFIRI, FOLFIRI-BEVACIZUMAB, FOLFIRI-CETUXIMAB, FOLFIRINOX, FOLFOX, Folotyn® (Pralatrexate), FU-LV, Fulvestrant, Gardasil® (Recombinant HPV Quadrivalent Vaccine), Gardasil 9® (Recombinant HPV Nonavalent Vaccine), Gazyva® (Obinutuzumab), Gefitinib, Gemcitabine Hydrochloride, GEMCITABINE-CISPLATIN, GEMCITABINE-OXALIPLATIN, Gemtuzumab Ozogamicin, Gemzar® (Gemcitabine Hydrochloride), Gilotrif® (Afatinib Dimaleate), Gleevec® (Imatinib Mesylate), Gliadel® (Carmustine Implant), Gliadel Wafer® (Carmustine Implant), Glucarpidase, Goserelin Acetate, Halaven® (Eribulin Mesylate), Hemangeol® (Propranolol Hydrochloride), Herceptin® (Trastuzumab), HPV Bivalent Vaccine, Recombinant, HPV Nonavalent Vaccine, Recombinant, HPV Quadrivalent Vaccine, Recombinant, Hycamtin® (Topotecan Hydrochloride), Hydrea® (Hydroxyurea), Hydroxyurea, Hyper-CVAD, Ibrance® (Palbociclib), Ibritumomab Tiuxetan, Ibrutinib, ICE, Iclusig® (Ponatinib Hydrochloride), Idamycin® (Idarubicin Hydrochloride), Idarubicin Hydrochloride, Idelalisib, Idhifa® (Enasidenib Mesylate), Ifex® (Ifosfamide), Ifosfamide, Ifosfamidum® (Ifosfamide), IL-2 (Aldesleukin), Imatinib Mesylate, Imbruvica® (Ibrutinib), Imfinzi® (Durvalumab), Imiquimod, Imlygic® (Talimogene Laherparepvec), Inlyta® (Axitinib), Inotuzumab Ozogamicin, Interferon Alfa-2b, Recombinant, Interleukin-2 (Aldesleukin), Intron A® (Recombinant Interferon Alfa-2b), Iodine I 131 Tositumomab and Tositumomab, Ipilimumab, Iressa® (Gefitinib), Irinotecan Hydrochloride, Irinotecan Hydrochloride Liposome, Istodax® (Romidepsin), Ixabepilone, Ixazomib Citrate, Ixempra® (Ixabepilone), Jakafi® (Ruxolitinib Phosphate), JEB, Jevtana® (Cabazitaxel), Kadcyla® (Ado-Trastuzumab Emtansine), Keoxifene® (Raloxifene Hydrochloride), Kepivance® (Palifermin), Keytruda® (Pembrolizumab), Kisqali® (Ribociclib), Kymriah® (Tisagenlecleucel), Kyprolis® (Carfilzomib), Lanreotide Acetate, Lapatinib Ditosylate, Lartruvo® (Olaratumab), Lenalidomide, Lenvatinib Mesylate, Lenvima® (Lenvatinib Mesylate), Letrozole, Leucovorin Calcium, Leukeran® (Chlorambucil), Leuprolide Acetate, Leustatin® (Cladribine), Levulan® (Aminolevulinic Acid), Linfolizin® (Chlorambucil), LipoDox® (Doxorubicin Hydrochloride Liposome), Lomustine, Lonsurf® (Trifluridine and Tipiracil Hydrochloride), Lupron® (Leuprolide Acetate), Lupron Depot® (Leuprolide Acetate), Lupron Depot-Ped® (Leuprolide Acetate), Lynparza® (Olaparib), Marqibo® (Vincristine Sulfate Liposome), Matulane® (Procarbazine Hydrochloride), Mechlorethamine Hydrochloride, Megestrol Acetate, Mekinist® (Trametinib), Melphalan, Melphalan Hydrochloride, Mercaptopurine, Mesna, Mesnex® (Mesna), Methazolastone® (Temozolomide), Methotrexate, Methotrexate LPF® (Methotrexate), Methylnaltrexone Bromide, Mexate® (Methotrexate), Mexate-AQ® (Methotrexate), Midostaurin, Mitomycin C, Mitoxantrone Hydrochloride, Mitozytrex® (Mitomycin C), MOPP, Mozobil® (Plerixafor), Mustargen® (Mechlorethamine Hydrochloride), Mutamycin® (Mitomycin C), Myleran® (Busulfan), Mylosar® (Azacitidine), Mylotarg® (Gemtuzumab Ozogamicin), Nanoparticle Paclitaxel® (Paclitaxel Albumin-stabilized Nanoparticle Formulation), Navelbine® (Vinorelbine Tartrate), Necitumumab, Nelarabine, Neosar® (Cyclophosphamide), Neratinib Maleate, Nerlynx® (Neratinib Maleate), Netupitant and Palonosetron Hydrochloride, Neulasta® (Pegfilgrastim), Neupogen® (Filgrastim), Nexavar® (Sorafenib Tosylate), Nilandron® (Nilutamide), Nilotinib, Nilutamide, Ninlaro® (Ixazomib Citrate), Niraparib Tosylate Monohydrate, Nivolumab, Nolvadex® (Tamoxifen Citrate), Nplate® (Romiplostim), Obinutuzumab, Odomzo® (Sonidegib), OEPA, Ofatumumab, OFF, Olaparib, Olaratumab, Omacetaxine Mepesuccinate, Oncaspar® (Pegaspargase), Ondansetron Hydrochloride, Onivyde® (Irinotecan Hydrochloride Liposome), Ontak® (Denileukin Diftitox), Opdivo® (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat® (Carboplatin), Paraplatin® (Carboplatin), Pazopanib Hydrochloride, PCV, PEB, Pegaspargase, Pegfilgrastim, Peginterferon Alfa-2b, PEG-Intron® (Peginterferon Alfa-2b), Pembrolizumab, Pemetrexed Disodium, Perjeta® (Pertuzumab), Pertuzumab, Platinol® (Cisplatin), Platinol-AQ® (Cisplatin), Plerixafor, Pomalidomide, Pomalyst® (Pomalidomide), Ponatinib Hydrochloride, Portrazza® (Necitumumab), Pralatrexate, Prednisone, Procarbazine Hydrochloride, Proleukin® (Aldesleukin), Prolia® (Denosumab), Promacta® (Eltrombopag Olamine), Propranolol Hydrochloride, Provenge® (Sipuleucel-T), Purinethol® (Mercaptopurine), Purixan® (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor® (Methylnaltrexone Bromide), R-EPOCH, Revlimid® (Lenalidomide), Rheumatrex® (Methotrexate), Ribociclib, R-ICE, Rituxan® (Rituximab), Rituxan Hycela® (Rituximab and Hyaluronidase Human), Rituximab, Rituximab and, Hyaluronidase Human, Rolapitant Hydrochloride, Romidepsin, Romiplostim, Rubidomycin® (Daunorubicin Hydrochloride), Rubraca® (Rucaparib Camsylate), Rucaparib Camsylate, Ruxolitinib Phosphate, Rydapt® (Midostaurin), Sclerosol Intrapleural Aerosol (Talc), Siltuximab, Sipuleucel-T, Somatuline Depot® (Lanreotide Acetate), Sonidegib, Sorafenib Tosylate, Sprycel® (Dasatinib), STANFORD V, Sterile Talc Powder (Talc), Steritalc® (Talc), Stivarga® (Regorafenib), Sunitinib Malate, Sutent® (Sunitinib Malate), Sylatron® (Peginterferon Alfa-2b), Sylvant® (Siltuximab), Synribo Synribo® (Omacetaxine Mepesuccinate), Tabloid® (Thioguanine), TAC, Tafinlar® (Dabrafenib), Tagrisso® (Osimertinib), Talc, Talimogene Laherparepvec, Tamoxifen Citrate, Tarabine PFS® (Cytarabine), Tarceva® (Erlotinib Hydrochloride), Targretin® (Bexarotene), Tasigna® (Nilotinib), Taxol® (Paclitaxel), Taxotere® (Docetaxel), Tecentriq® (Atezolizumab), Temodar® (Temozolomide), Temozolomide, Temsirolimus, Thalidomide, Thalomid® (Thalidomide), Thioguanine, Thiotepa, Tisagenlecleucel, Tolak® (Fluorouracil—Topical), Topotecan Hydrochloride, Toremifene, Torisel® (Temsirolimus), Tositumomab and Iodine I 131 Tositumomab, Totect® (Dexrazoxane Hydrochloride), TPF, Trabectedin, Trametinib, Trastuzumab, Treanda® (Bendamustine Hydrochloride), Trifluridine and Tipiracil Hydrochloride, Trisenox® (Arsenic Trioxide), Tykerb® (Lapatinib Ditosylate), Unituxin® (Dinutuximab), Uridine Triacetate, VAC, Vandetanib, VAMP, Varubi® (Rolapitant Hydrochloride), Vectibix® (Panitumumab), VeIP, Velban® (Vinblastine Sulfate), Velcade® (Bortezomib), Velsar® (Vinblastine Sulfate), Vemurafenib, Venclexta® (Venetoclax), Venetoclax, Verzenio® (Abemaciclib), Viadur® (Leuprolide Acetate), Vidaza® (Azacitidine), Vinblastine Sulfate, Vincasar PFS® (Vincristine Sulfate), Vincristine Sulfate, Vincristine Sulfate Liposome, Vinorelbine Tartrate, VIP, Vismodegib, Vistogard® (Uridine Triacetate), Voraxaze® (Glucarpidase), Vorinostat, Votrient® (Pazopanib Hydrochloride), Vyxeos® (Daunorubicin Hydrochloride and Cytarabine Liposome), Wellcovorin® (Leucovorin Calcium), Xalkori® (Crizotinib), Xeloda® (Capecitabine), XELIRI, XELOX, Xgeva® (Denosumab), Xofigo® (Radium 223 Dichloride), Xtandi® (Enzalutamide), Yervoy® (Ipilimumab), Yondelis® (Trabectedin), Zaltrap® (Ziv-Aflibercept), Zarxio® (Filgrastim), Zejula® (Niraparib Tosylate Monohydrate), Zelboraf® (Vemurafenib), Zevalin® (Ibritumomab Tiuxetan), Zinecard® (Dexrazoxane Hydrochloride), Ziv-Aflibercept, Zofran® (Ondansetron Hydrochloride), Zoladex® (Goserelin Acetate), Zoledronic Acid, Zolinza® (Vorinostat), Zometa® (Zoledronic Acid), Zydelig® (Idelalisib), Zykadia® (Ceritinib), and/or Zytiga® (Abiraterone Acetate). The treatment methods can include or further include checkpoint inhibitors including, but are not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, BI754111, Sym022, FS118, MGD013, and Immutep).

It is understood and herein contemplated that adoptive immune cell immunotherapy (such as, for example, CAR T cells or TILs) can be used to treat autoimmune diseases. As used herein, “autoimmune disease” refers to a set of diseases, disorders, or conditions resulting from an adaptive immune response (T cell and/or B cell response) against the host organism. In such conditions, either by way of mutation or other underlying cause, the host T cells and/or B cells and/or antibodies are no longer able to distinguish host cells from non-self-antigens and attack host cells bearing an antigen for which they are specific. Examples of autoimmune diseases include, but are not limited to graft versus host disease, transplant rejection, Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Bald disease, Behcet's disease, Benign mucosal pemphigoid, Bickerstaffs encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammaglobulinemia, IgA Nephropathy, IgG4-related sclerosing disease, Immune thrombocytopenic purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)). In one aspect, disclosed herein are methods of treating, inhibiting, reducing, decreasing, ameliorating and/or preventing an autoimmune disease in a subject comprising administering to the subject an adoptive immune cell immunotherapy (including, but not limited to administration of chimeric antigen receptor (CAR) T cells, CAR natural killer (NK) cells, tumor infiltrating lymphocytes (TILs)) and an agent that blocks dysregulation of the citric acid cycle (such as, for example, an agent that inhibits inducible nitric oxide synthase (iNOS) and/or an agent that inhibits immune responsive gene 1 (IRG1)). In one aspect, the immune cells in the adoptive immune cell immunotherapy are CD8+ T cells, CD4+ T cells, NK cells, or NK T cells.

In one aspect, disclosed herein are methods of treating, inhibiting, reducing, decreasing, ameliorating, and/or preventing an autoimmune disease of any preceding aspect, wherein the iNOS inhibitor and/or IRG1 inhibitor is an antibody, antibody fragment, small molecule (such as, for example, N⁶-(1-Iminoethyl)-lysine, hydrochloride (L-NIL), siRNA, or oligonucleotide that disrupts iNOS transcription, expression, or binding.

Also disclosed herein are methods of treating, inhibiting, reducing, decreasing, ameliorating, and/or preventing an autoimmune disease in a subject comprising administering to the subject an adoptive immune cell immunotherapy (including, but not limited to administration of chimeric antigen receptor (CAR) T cells, CAR natural killer (NK) cells, tumor infiltrating lymphocytes (TILs)) wherein the immune cell has been modified to disrupt expression of IRG1. In one aspect, the immune cells in the adoptive immune cell immunotherapy are CD8+ T cells, CD4+ T cells, NK cells, or NK T cells.

In one aspect, disclosed herein are methods of treating, inhibiting, reducing, decreasing, ameliorating, and/or preventing an autoimmune disease, wherein the expression of iNOS and/or IRG1 is disrupted by a clustered regularly interspaced short palindromic repeat (CRISPR)/CRISPR-associated (Cas)integration systems (such as, for example a Class 2 CRISPR/Cas integration system, including by not limited to a CRISPR/Cas9 integration system) that targets iNOS or IRG1. In some aspects, the immune cell is modified ex vivo. In general, “CRISPR system” or “CRISPR integration system” refers collectively to transcripts and other elements involved in the expression of or directing the activity of CRISPR-associated “Cas” genes. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

Also disclosed herein are methods of rescuing non-durable responses (NDR) to an adoptive immune cell immunotherapy for a cancer or autoimmune disease in a subject comprising a) obtaining previously adoptively transferred immune cells from the subject or macrophage from tumor microenvironment (TME) of the subject, b) measuring the expression level of immune responsive gene 1 (IRG1) in the transferred immune cell or inducible nitric oxide synthase (iNOS) in the macrophage; and c) administering to the subject an agent that inhibits IRG1 and/or iNOS or administering to the subject chimeric antigen receptor (CAR) immune cell comprising a disrupted iNOS gene and/or IRG1 gene when the expression level of IRG1 in the adoptively transferred immune cell has increased relative to a control or the expression level of iNOS in the macrophage from the TME has increased relative to a control.

### EXAMPLES

### Example 1: CAR T Cell-Driven Induction of iNOS in Tumor-Associated Macrophages Promotes CAR T Cell Resistance in B Cell Lymphoma

Disclosed herein is a study which reports the reciprocal interactions between CAR T cells and macrophages on the development of resistance to CAR T cell therapy in B cell lymphoma. The study found that IFN-γ produced by CAR T cells induces phenotypic alterations in macrophages, increasing their immunoregulatory potential. The M2-like macrophages expressing arginase-1 (ARG-1) are transformed into iNOS⁺ ARG-1⁺ co-expressing suppressive macrophages, impairing CAR T cell effector function and metabolism. Inhibition of iNOS, in combination with CAR T cell treatment, enhances the survival of B cell tumor-bearing mice. These findings demonstrate a key counter-regulatory mechanism induced by IFN-γ-producing CAR T cells that restricts their anti-cancer efficacy and highlight therapeutic opportunities to overcome macrophage-mediated CAR T cell dysfunction and improve patient outcomes.

### Materials and Methods

Patient samples: All samples were prospectively obtained from patients with relapsed or refractory LBCL who underwent axi-cel treatment at H. Lee Moffitt Cancer Center. The collection of samples was conducted in accordance with approved protocols by the institutional review board. Pre-treatment tumor biopsies were obtained within 1 month prior to axi-cel infusion and before lymphodepletion. Patients who achieved sustained remission for at least 6 months following axi-cel infusion were classified as durable responders (DR). Non-durable responders (NDR) were patients who either experienced lymphoma relapse or passed away due to any cause.

Mice: All animal studies were performed according to a protocol approved by H. Lee Moffitt Cancer Center and Research Institute and the University of South Florida Institutional Animal Care and Use Committee. C57BL/6J mice, Nos2^{-/-} (B6.129P2-Nos^{2tm1Lau}/J) mice, Ifng^{-/-} (B6.129S7-Ifng^{tm1Ts}/J) mice, and Rag1^{+/+} mice (B6.129S7-Rag1^{tm1Mom}/J) were purchased from Jackson Laboratories. Rag1^{-/-} mice were bred in-house.

Cell lines: Eμ-myc cells were derived from the axillary lymph node of tumor-bearing Eμ-myc transgenic mice, a spontaneous Burkitt-like lymphoma model. For some experiments, Eμ-myc cells that were retrovirally transduced to express GFP-firefly luciferase (Eμ-myc-GFP-FFL) were used. Eμ-myc cells were maintained on irradiated (30 Gy) NIH-3T3 feeder cells in RPMI-1640/IMDM (1/1, v/v) supplemented with 10% heat-inactivated fetal bovine serum (HI-FBS), 2 mM L-glutamine, 100 U/ml Penicillin/Streptomycin, and 22.5 μM β-mercaptoethanol. Prior to use as feeder cells, NIH/3T3 was maintained in DMEM supplemented with 10% HI-FBS, 2 mM L-glutamine, and 100 U/ml Penicillin/Streptomycin. Cell lines were routinely tested for the absence of mycoplasma contamination using the Universal Mycoplasma Detection kit (ATCC) or MycoAlert PLUS mycoplasma detection kit (Lonza).

Genomics: RNA-sequencing was performed as previously described 20,62. Formalin-fixed paraffin-embedded (FFPE) or snap-frozen samples were obtained and examined by a hematologist for tumor content. RNA was extracted and RNA-sequencing libraries were prepared using NuGen RNA-Seq Multiplex System (Tecan US) according to the manufacturer's protocols. The libraries were then sequenced on the Illumina NextSeq 500 system with a 75-base paired-end run at 80 to 100 million read pairs per sample. To determine the immune cell composition in bulk RNA-seq profiles of tumor biopsies, CIBERSORTx v.1.0.41 (https://cibersortx.stanford.edu) with the LM22 signature matrix was applied. Geneset enrichment analysis of M2-associated gene expression was performed on the R package GSVA, utilizing a panel of genes as previously described.

Multiplex immunofluorescence: Multiplex immunofluorescence staining was performed as previously described. FFPE tumor biopsies were obtained and examined by a hematopathologist for tumor content. Tissue microarray (TMA) including 21 cores was created from a total of 11 patients (9 cores from 6 patients with DR, 12 cores from 5 patients with NDR). The TMA was then subjected to staining by OPAL multiplexing method, based on Tyramide Signal Amplification (TSA) on the Leica BOND™ Automated Stainer (Leica Biosystems, Wetzlar, Germany), using two sets of antibodies against specific markers: 1) CD68 (CST, D4B9C, HIER—Citrate pH 6.0, 1:100, dye520), PAX5 (Abcam, EPR3730(2), HIER—EDTA pH 9.0, 1:400, dye690), CD4 (Cell Marque, EP204, HIER—EDTA pH 9.0, 1:100, dye570), CD163 (Abcam, OTI2G12, HIER—Citrate pH 6.0, 1:50, dye540), FOXP3 (Abcam, 236A/E7, HIER—EDTA pH 9.0, 1:200, dye620), and CD3 (Thermo Fisher, SP7, HIER—EDTA pH 9.0, 1:400, dye650). After the final stripping step, DAPI counterstain is applied to the multiplexed slide and is removed from BOND RX for coverslipping with ProLong Diamond Antifade Mountant (ThermoFisher Scientific). One additional Fluorescent Multiplex-IF panels were designed using there following antibodies: 2) iNOS (Thermo Fisher, 4E5, HIER—EDTA pH 9.0, 1:100, dye620) and CD14 (Abcam, LPSR/2386, HIER—EDTA pH 9.0, 1:300, dye570). All slides were imaged with the Vectra™3 Automated Quantitative Pathology Imaging System. Images were analyzed using HALO Image Analysis Platform (Indica Labs, Albuquerque, NM).

Generation of retroviral constructs: Plasmids encoding 19dz and 1928z CAR constructs in SFG γ-retroviral vectors have been described previously. Briefly, 1928z CAR construct includes anti-murine CD19 scFv (1D3), murine CD8α transmembrane and hinge domains, murine CD28 intracellular domain, and murine CD3z intracellular domain followed by the mCherry reporter via glycine-serine linker. 19dz CAR construct includes the same sequence as 1928z construct except for absence of CD28 intracellular domain and having a truncated CD3z intracellular domain. For retrovirus production, plasmids were transfected to H29 cell lines using a calcium phosphate transfection kit (Invitrogen) to produce vesicular stomatitis virus G-glycoprotein-pseudotyped retroviral supernatants. These retroviral supernatants were subsequently transduced to Phoenix-ECO cell lines to stably generate Moloney murine leukemia virus-pseudotyped retroviral particles.

Mouse T cell isolation and CAR T cell generation: Mouse spleens were excised, mechanically disrupted, and filtered through a 40 m cell strainer. CD3+ T cells were enriched via negative selection using EasySep Mouse T Cell Isolation Kit (STEMCELL Technologies). T cells were activated and expanded with anti-CD3/28 Dynabeads (Gibco) at a bead-to-cell ratio of 0.8:1. T cells were spinoculated (2000×g, 1 h, room temperature) twice, 24 h and 48 h after initial T cell activation, with viral supernatants collected from Phoenix-ECO cells on retronectin (Takara) coated plates. Following the second spinoculation, T cells were maintained for one day. On day 5, anti-CD3/28 Dynabeads were removed, and CAR T cells were used for in vitro or in vivo experiments. CAR transduction efficiency was determined by flow cytometry as a percentage of mCherry⁺ cells in live cells. Mouse T cells were cultured in RPMI-1640 supplemented with 10% HI-FBS, 2 mM L-glutamine, 100 U/ml Penicillin/Streptomycin, 1× nonessential amino acids, 1 mM sodium pyruvate, 10 mM HEPES, 55 μM 2-mercaptoethanol, and 100 IU/ml recombinant human IL-2.

Animal experiment: Six- to 10-week-old Rag1^{+/+} mice of both sexes were intraperitoneally (i.p.) injected with 3×10⁶ Eμ-myc-GFP-FFL cells to generate tumors localized in peritoneal cavity. Tumor engraftment was verified by bioluminescence imaging one day before CAR T cell transfer. Mice were randomized to different treatment groups without differences in pre-treatment tumor load. Seven days after tumor cell inoculation, mice were injected i.p. with 5×10⁶ CAR T cells in 300 μl PBS. For survival experiments, L-NIL or PBS was administered i.p. once per day at 20 mg/kg body weight starting on the same day of tumor cell injection. Experimental endpoints were achieved when mice demonstrated signs of morbidity or hind-limb paralysis, or when solid tumor masses reached 2000 mm³ for some mice that developed palpable masses. Bioluminescence imaging was performed by IVIS Lumina III In Vivo Imaging System (PerkinElmer) with Living Image software (PerkinElmer).

Macrophage development and polarization: BMDMs were generated from bone marrow cells harvested from femurs and tibias of WT or iNOS^{-/-} mice. Following red blood cell lysis by ACK (Ammonium-Chloride-Potassium) lysis buffer, 1×10⁷ bone marrow cells were cultured in 10-cm tissue culture dish in 10 ml of RPMI-1640 supplemented with 10% HI-FBS, 2 mM L-glutamine, 100 U/ml Penicillin/Streptomycin, and 20 ng/ml M-CSF (R&D systems) for 7 days. On day 3, 10 ml of fresh medium with 20 ng/ml M-CSF was added. On day 5, the culture medium was entirely discarded and replaced by 15 ml of fresh medium with 20 ng/ml M-CSF. On day 6, BMDMs were activated for 24 h with 20 ng/ml of IL-4 and IL-10 (Peprotech) to develop imMac or cultured in media only to use as unMac. M-CSF (20 ng/ml) was added during activation with cytokines. On day 7, adherent cells were harvested by gentle scraping and used for in vitro experiments.

Mouse peritoneal cells: Peritoneal cells were obtained by peritoneal lavage as previously described. After euthanizing mice, 5 ml ice-cold PBS/2 mM EDTA were i.p. injected. Bellies were massaged for one minute, and subsequently incised to drain the lavage fluid in a collection tube. Cells were filtered through a 40 m cell strainer. Following red blood cell lysis with ACK lysing buffer, peritoneal cells were used for subsequent analyses. For ex vivo coculture experiments with CAR T cells, EasySep Mouse CD11b Positive Selection Kit II (STEMCELL Technologies) was used to isolate CD11b⁺ myeloid cells.

Expansion assay: The expansion of mCherry⁺ CAR T cells was determined by an IncuCyte S3 live cell analysis system (Essen Bioscience). 2×10⁴ Eμ-myc cells and 2×10⁴ CAR T cells were cocultured in the absence or presence of 0.5×10⁴ macrophages (CAR T:Eμ-myc cell:Macrophage=1:1:0.25, unless otherwise indicated in the figures) in a 96-well black-walled clear bottom plate in 120 μl of media. Cell images were captured at 4× magnification. The expansion index was calculated by dividing the total integrated red intensity (RCU×m²/mm²) at each time point by the first time point.

Griess assay: 2×10⁴ Eμ-myc cells and 2×10⁴ CAR T cells were cocultured in the presence or absence of 0.5×10⁴ macrophages in a 96-well plate in 120 μl of media. Coculture supernatants were harvested, and nitric oxide levels were measured using Griess reagent system (Promega) according to manufacturer's instructions. Absorbance was read at 560 nm using microplate reader (GloMax, Promega), and NO₂ concentrations were determined by standard curve. Standard curve was prepared with diluting 0.1M sodium nitrite standard (provided in the kit) with the culture media used for experiments.

BrdU incorporation assay: 2×10⁵ Eμ-myc cells and 2×10⁵ CART cells were cocultured in the absence or presence of 0.5×10⁵ macrophages in a 24-well plate in 1200 μl of media. At 24 h of coculture, BrdU was added to each well at 10 μM. After an additional incubation for 18 h, cells were harvested. BrdU staining was performed according to APC BrdU flow kit (BD Pharmingen) and BrdU incorporation was analyzed by flow cytometry.

Flow cytometry: The following fluorophore-conjugated anti-mouse antibodies were used. From BD Horizon: anti-CD45 (30-F11), anti-CD19 (1D3), anti-CD11b (M1/70), and anti-CD3ε (145-2C11). From BioLegend: anti-CD8α (53-6.7), anti-PD-L1 (1° F.9G2), and anti-F4/80 (BM8). From eBioscience: anti-mouse ARG-1 (AlexF5) and anti-NOS2 (CXNFT). Fc receptors were blocked using FcR Blocking Reagent (anti-mouse CD16/CD32 antibody, Invitrogen). DAPI (BD Pharmingen) and Zombie NIR Fixable Viability Kit (BioLegend) were used as viability dyes. For intracellular staining, surface-labeled cells were fixed and permeabilized with Cytofix/Cytoperm kit (BD Biosciences) according to the manufacturer's instructions and then stained with intracellular antibodies. For cell surface CAR staining, protein L-biotin conjugate followed by PE-conjugated streptavidin was used. Flow cytometry was performed on a LSR II or FACSymphony instrument (BD Biosciences). Data were analyzed with the FlowJo software (FlowJo LLC).

CAR T cell isolation from initial coculture for subsequent downstream assays: 1×10⁶ CAR T cells and 1×10⁶ Eμ-myc-GFP-FFL cells were cocultured in the absence or presence of 0.25×10⁶ macrophages in a 6-well plate in 6 ml of media for 48 h. After initial coculture, cells were harvested, and T cells were isolated using Mouse T Cell Isolation Kit (STEMCELL Technologies). T cell purity was 100% as tested by flow cytometry. Percentage of CAR-expressing T cells was determined with flow cytometry and were subsequently used for downstream assays.

Luciferase-based killing assay: 2×10⁴ Eμ-ALL-GFP-FFL cells were cocultured with CAR T cells at different effector-to-target ratios in a 96-well white-walled plate in 100 μl of media. Following incubation, 100 μl luciferase substrate reagent (ONE-Glo Luciferase assay system, Promega) was added to each well. Target cells alone were plated at the same cell density to determine maximum luciferase signals. Emitted luminescence was detected in the microplate reader (GloMax, Promega). Percent lysis was determined as (1−sample signal/maximum signal)×100.

Cytokine secretion assay: 2×10⁴ Eμ-myc cells were cocultured with 2×10⁴ CAR T cells in a 96-well plate in a total volume of 100 μl of media. Supernatants were collected and analyzed for IFN-γ and TNF-α secretion using Ella automated immunoassay system (Proteinsimple Bio-techne) according to manufacturer's instructions.

Immunoblotting: Cells were lysed in ELB lysis buffer (50 mM HEPES, pH 7.5, 250 mM NaCl, 5 mM EDTA, 0.5 mM DTT, 0.1% NP-40 alternative, 10 μg/ml aprotinin, 10 μg/ml leupeptin, and 100 μg/ml trypsin/chymotrypsin inhibitor). Following protein quantification with the Pierce BCA protein assay (ThermoFisher), the samples were mixed with a loading buffer containing 2-mercaptoethanol. The proteins were electrophoresed in 4-20% Tris-Glycine gels (Novex-Invitrogen) and transferred to PVDF membrane with a Bio-Rad Trans-Blot SD Semi-Dry Transfer Cell. The membrane was blocked in 5% bovine serum albumin (BSA) in TBST and subsequently blotted with primary and secondary antibodies in 5% BSA in TBST. The following antibodies were used: IDH1 (clone D2H1; Cell signaling, #8137S), IDH2 (clone D2E3B; Cell signaling, #56439S), IRG1 (clone E5B2G; Cell signaling, #19857S), 3-actin (clone AC-74, Sigma-Aldrich, #A2228), and horseradish peroxidase-conjugated secondary antibodies (Donkey-anti-Rabbit, Cytiva, #NA934-1ML); Sheep-anti-Mouse, Cytiva, #NA931-1ML). Membranes were imaged with a ChemiDoc Imaging System (BioRad, #17001401) and exported through ImageLab (BioRad #12012931).

Metabolomics and ¹³C₆-labeled glucose tracing analyses: For global metabolomics analysis of cell-cultured medium, the cell-free medium was obtained by performing rapid centrifugation (17,000×g, 10 sec, room temperature) to collect the supernatant. The metabolites present in 20 μl of the cell-cultured medium were then extracted using 80 μl of ice-cold MeOH. Following a 30 min incubation on ice and subsequent centrifugation (17,000×g, 20 min, 4° C.), the supernatant was subjected to LC-HRMS analysis.

Global metabolomic profiling and 13C6-labeled glucose tracing of CAR T cells, 1×10⁶ T cells were resuspended in either RPMI-1640 medium (RPMI+10% heat-inactivated dialyzed FBS) or ¹³C6-glucose substituted RPMI-1640 medium (glucose-free RPMI+10% heat-inactivated dialyzed FBS+11.1 mM ¹³C6-glucose). After 4 h incubation, cells were collected, rapidly centrifuged (17,000×g, 10 sec, room temperature), and medium was removed. T cells were washed with 1 ml of ice-cold PBS, and metabolites were extracted with 300 μl of 80% methanol via incubation at −80° C. for 15 min. Samples were centrifuged (17,000×g, 20 min, 4° C.), and supernatants were transferred to an Eppendorf tube and dried in a vacuum evaporator overnight. The dried extracts were resuspended in 20 μl of aqueous 50% methanol, clarified by centrifugation (17,000×g, 20 min, room temperature), and analyzed by LC-HRMS.

LC-HRMS analysis was performed on a Vanquish UPLC coupled with a Q-Exactive HF mass spectrometer, employing the same conditions as the previously established methods 76. A ZIC-pHILIC LC column (4.6 mm inner diameter×150 mm length, 5 m particle size, MilliporeSigma, Burlington, MA) with a ZIC-pHILIC guard column (4.6 mm inner diameter×20 mm length, MilliporeSigma, Burlington, MA) was used for chromatographic separation at a column temperature of 30° C. The mobile phases included 10 mM (NH4)2CO3 and 0.05% NH40H in H2O for mobile phase A, and 100% can for acetonitrile (ACN) mobile phase B. The LC gradient conditions were as follows: 0 to 13 min: a decreasing of 80% to 20% of mobile phase B, 13 to 15 min: 20% of mobile phase B. The ionization was set to negative mode, with the MS scan range set to 60 to 1000 m/z. The mass resolution was 70,000, and the AGC target was 1×106. The sample loading volume was 5 μl. The unlabeled or ¹³C-labeled metabolite peaks were extracted using EL-Maven with a metabolite standard-based in-house library. For global metabolomic profiling, peak areas of metabolites were normalized by the median value of the total for identified metabolite peak areas in each sample. For the ¹³C-labeled metabolite peaks, the natural isotope peak area was corrected using IsoCor (Version 2.2).

Seahorse assay: ECAR and OCR were measured using a Seahorse Extracellular Flux Analyzer (Agilent Technologies). XF96 microplates were coated with CellTak a day before analyses. To assay glycolytic function, T cells were resuspended in glucose-free XF medium supplied with 2 mM L-glutamine and 1 mM sodium pyruvate and seeded at 2×10⁵ cells in 180 μl per well. Following incubation in a CO₂-free incubator for 60 min at 37° C. for pH stabilization, ECAR was measured in response to 10 mM glucose, 1 μM oligomycin, and 50 mM 2-deoxyglucose. To assay mitochondrial function, T cells were resuspended in XF medium supplied with 2 mM L-glutamine, 1 mM sodium pyruvate, and 10 mM glucose and seeded at 2×10⁵ cells in 180 μl per well. Following incubation in a CO₂-free incubator for 60 min at 37° C. for pH stabilization, OCR was measured in response to 1 μM oligomycin, 1 μM FCCP, and 0.5 μM rotenone and antimycin.

### Results

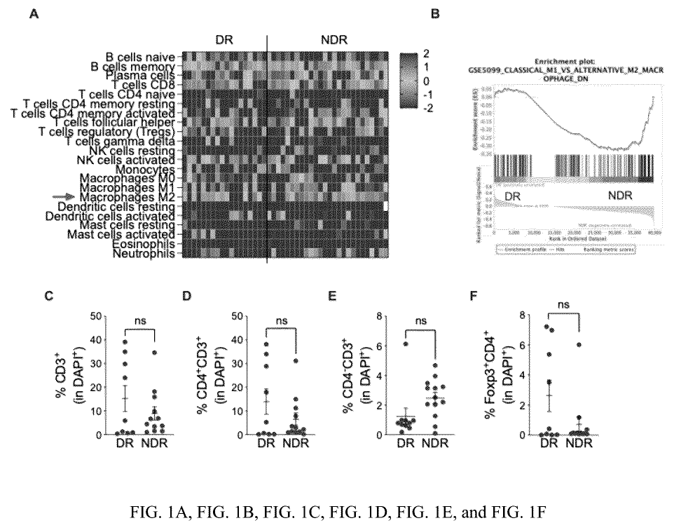

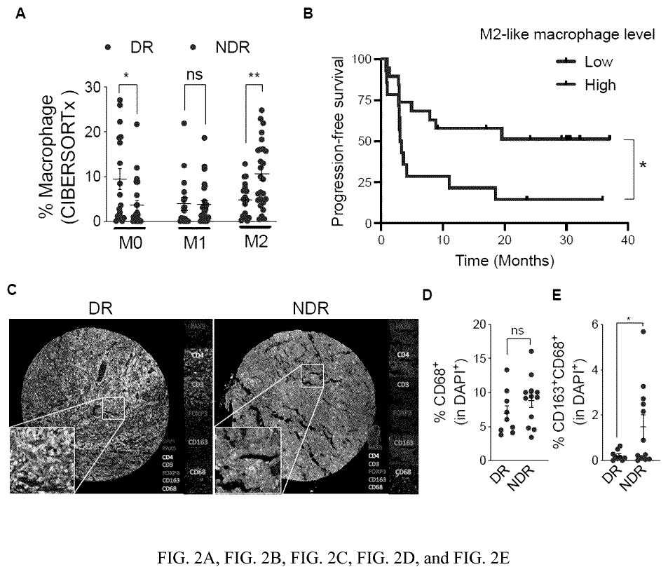

Macrophages in the pre-CAR T cell treatment TME are linked to therapeutic responses in LBCL patients: The study examined the tumor immune infiltrate and its relationship with clinical outcomes in patients with LBCL receiving Axicabtagene ciloleucel (axi-cel). Bulk RNA sequencing (RNA-seq) was performed on patient tumor biopsies taken before lymphodepletion and CAR T cell treatment. Subsequently, CIBERSORTx was used to deconvolute intratumoral immune cell composition (FIG. 1A). The study found that patients with non-durable responses (NDR) to CAR T cell therapy, characterized by lymphoma relapse or death from any cause, exhibited a higher proportion of transcriptionally identified M2-like macrophages compared to patients with durable responses (DR), who remained in remission for at least 6 months following axi-cel infusion (FIG. 2A). Similarly, gene set enrichment analysis (GSEA) revealed the enrichment of M2 macrophage-associated genes in patients with NDR (FIG. 1B). The proportion of nonactivated macrophages (M0) was lower in patients with NDR, while levels of M1-like macrophages were similar between patients with NDR and DR (FIG. 2A). Furthermore, it was observed that a higher abundance of M2-like macrophages in patients was associated with worse progression-free survival after axi-cel therapy (FIG. 2B).

Next, the study utilized multiplex immunofluorescence imaging to validate the proportions of tumor-associated immune populations in tissue microarrays including patient tumor specimens taken prior to lymphodepletion and CAR T cell therapy (FIG. 2C). The study determined that patients with NDR had a similar proportion of total CD68⁺ macrophages (FIG. 2D) but a significantly higher proportion of CD163⁺CD68⁺M2-like macrophages compared to patients with DR (FIG. 2E). Notably, the proportions of total CD3+ T cells, CD4⁺CD3+ T cells, CD4⁻CD3+ T cells, and FOXP3⁺CD4+ Tregs did not significantly correlate with NDR or DR status (FIG. 1C, FIG. 1D, FIG. 1E, FIG. 1F). These findings collectively indicate that the presence of M2-like macrophages within the TME prior to CAR T cell therapy is associated with poor therapeutic responses to axi-cel in patients with LBCL.

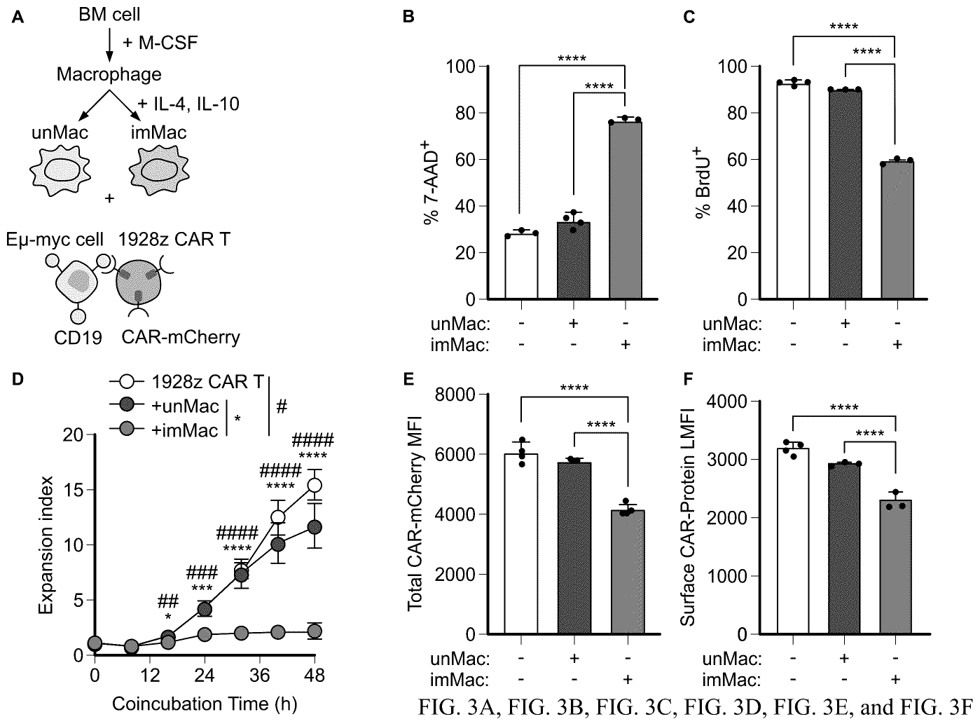

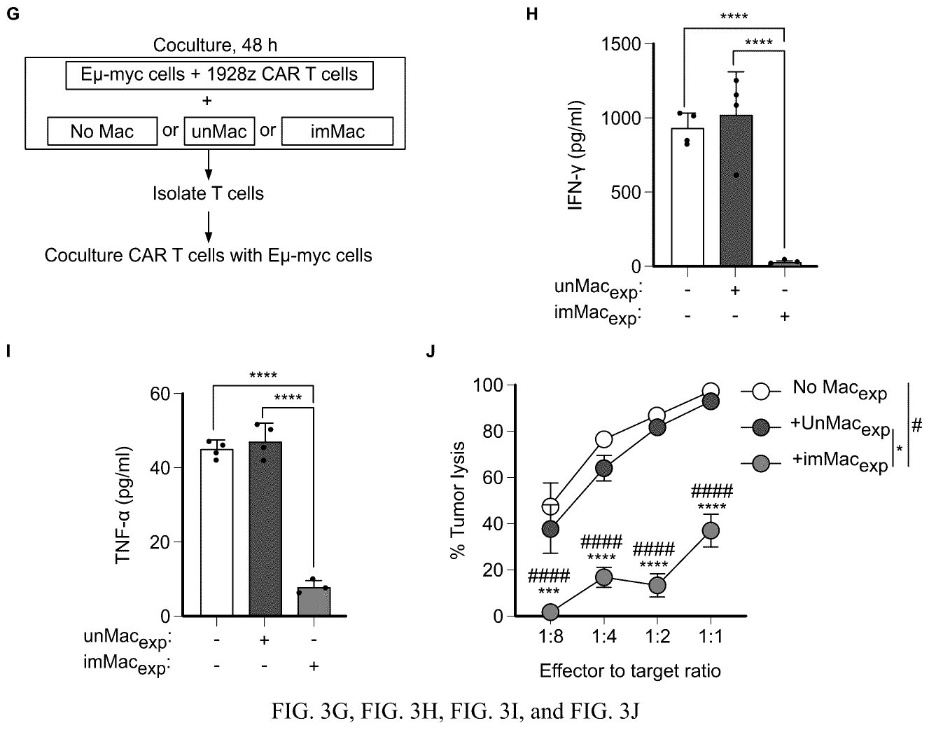

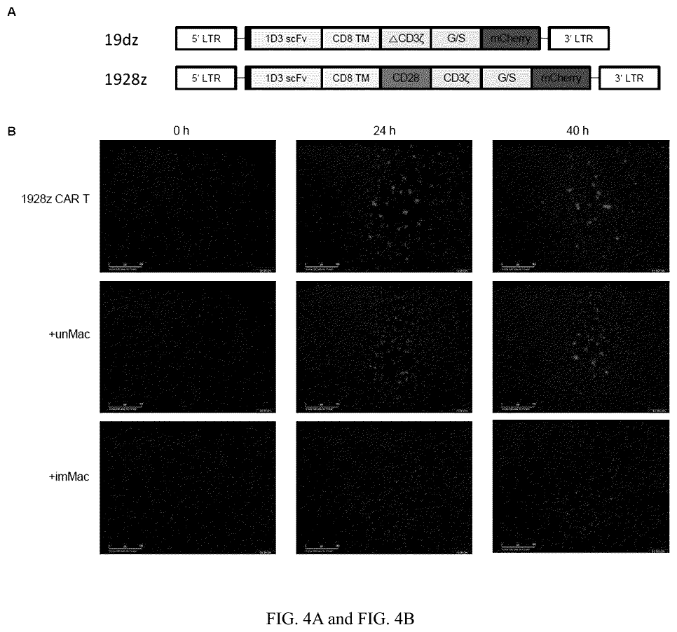

Immunoregulatory actions of macrophages on CAR T cells: To explore how M2-like macrophages may impact the cellular function of CAR T cells, the study employed a syngeneic coculture system (FIG. 3A). In this model, murine anti-CD19 CAR T cells, which include CD28 and CD3z signaling domains linked to a fluorescent mCherry reporter (1928z) (FIG. 4A), were cocultured with a murine malignant B cell line (Eμ-myc cells) in the presence or absence of mouse bone marrow-derived macrophages (BMDMs). The BMDMs were either unpolarized (unMac) or induced to differentiate into M2-like macrophages by culturing with type 2 cytokines IL-4 and IL-10. These differentiated M2-like macrophages were termed as ‘imMac’ to emphasize their distinctive immunoregulatory activity following coculture with CAR T cells. CAR T cells cocultured with imMac showed increased cell death (FIG. 3B) and reduced DNA replication (FIG. 3C) compared to CAR T cells cocultured with unMac or without macrophages. Correspondingly, CAR T cells exhibited diminished expansion during coculture with imMac (FIG. 3D, FIG. 4B). Moreover, CAR T cells cocultured with imMac showed lower total CAR expression (FIG. 3E) as well as reduced surface CAR expression (FIG. 3F). The study next explored the impact of imMac on CAR T cell effector function. To exclude the direct contribution of macrophage effector activities in these functional assays, CAR T cells, Eμ-myc cells, and macrophages were first cocultured for 48 hours (FIG. 3G). Next, CAR T cells isolated from the cocultures were evaluated for their effector function against fresh Eμ-myc cells. CAR T cells derived from cocultures with imMac exhibited impaired production of effector cytokines IFN-γ and tumor necrosis factor-alpha (TNF-α) (FIG. 3H, FIG. 3I) and demonstrated decreased ability to lyse target tumor cells (FIG. 3J). Collectively, these results show that macrophages polarized towards an M2-like phenotype exert immunoregulatory actions that impair multiple aspects of CAR T cell biology, including survival, expansion, and CAR-dependent effector functions.

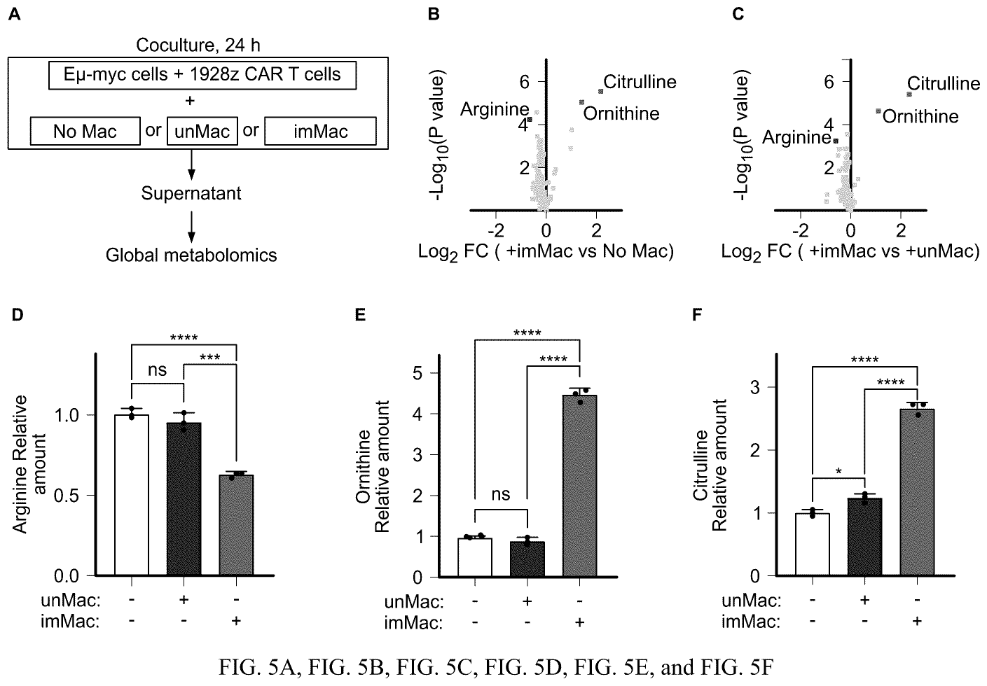

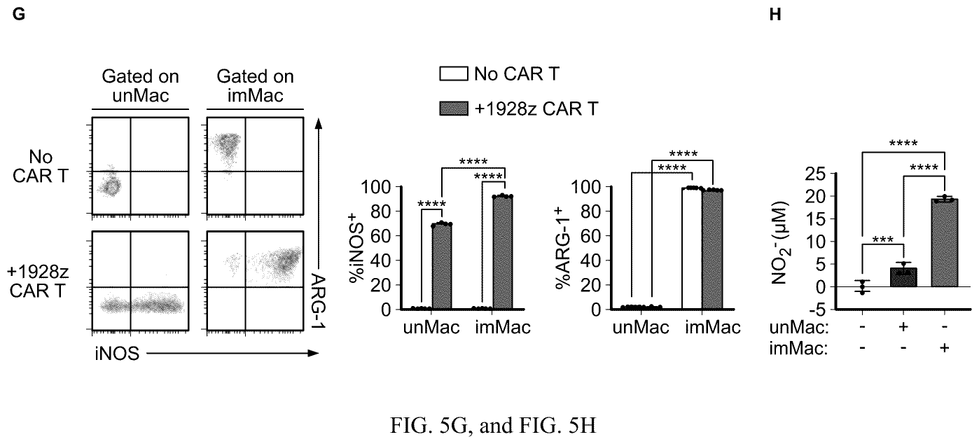

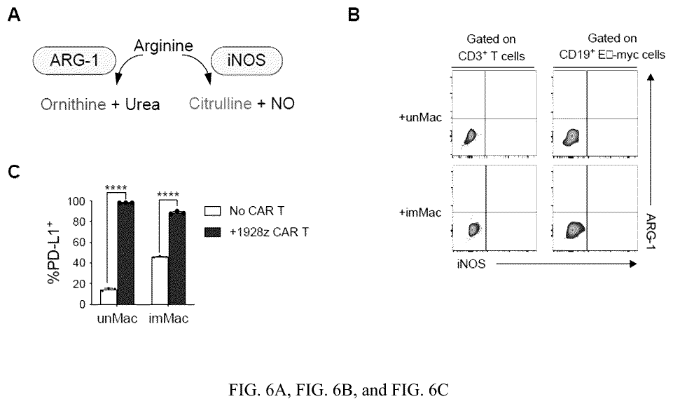

CAR T cell-exposed imMac upregulates iNOS: The study next interrogated the metabolic crosstalk between CAR T cells and imMac to investigate the potential involvement of immune-metabolic alterations. The study included a comprehensive analysis of metabolite profiles in the supernatants collected from the coculture model via global metabolomics using liquid chromatography-mass spectrometry (LC-MS) (FIG. 5A). The study identified a significant increase in citrulline and ornithine levels and a concomitant reduction in arginine levels within the supernatants derived from cocultures containing imMac compared to cocultures containing unMac or no macrophages (No Mac) (FIG. 5B, FIG. 5C, FIG. 5D, FIG. 5E, FIG. 5F). Macrophages possess the capacity to metabolize arginine through arginase-1 (ARG-1) or inducible nitric oxide synthase (iNOS) pathways, producing ornithine and urea or citrulline and nitric oxide (NO), respectively (FIG. 6A). It was observed that in the absence of CAR T cells, imMac exhibited high expression levels of ARG-1 but minimal expression of iNOS, whereas unMac displayed minimal expression of both ARG-1 and iNOS (FIG. 5G). When CAR T cells were cocultured with macrophages, there was a significant induction of iNOS expression in imMac and, to a lesser extent, in unMac. The expression levels of ARG-1 in unMac and imMac remained unchanged, regardless of the presence of CAR T cells in the cocultures. Neither CD3+ T cells nor Eμ-myc cells expressed ARG-1 or iNOS, confirming that the expression of these enzymes was limited to macrophages in this model (FIG. 6B). Consistent with the enhanced iNOS expression in imMac, higher levels of NO were produced in cocultures with imMac compared to cocultures with unMac or without macrophages (FIG. 5H). Additionally, coculture with CAR T cells substantially increased the expression of PD-L1 in both unMac and imMac (FIG. 6C). Together, these findings demonstrate that exposure of imMac to CAR T cells induces phenotypic changes in imMac, including enhanced arginine metabolism through the upregulation of iNOS.

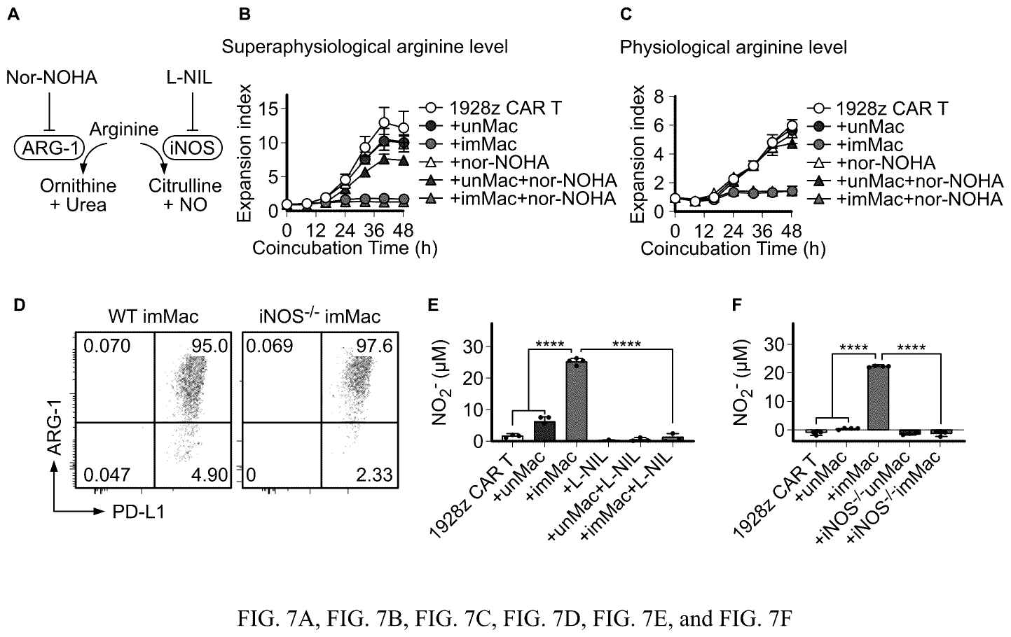

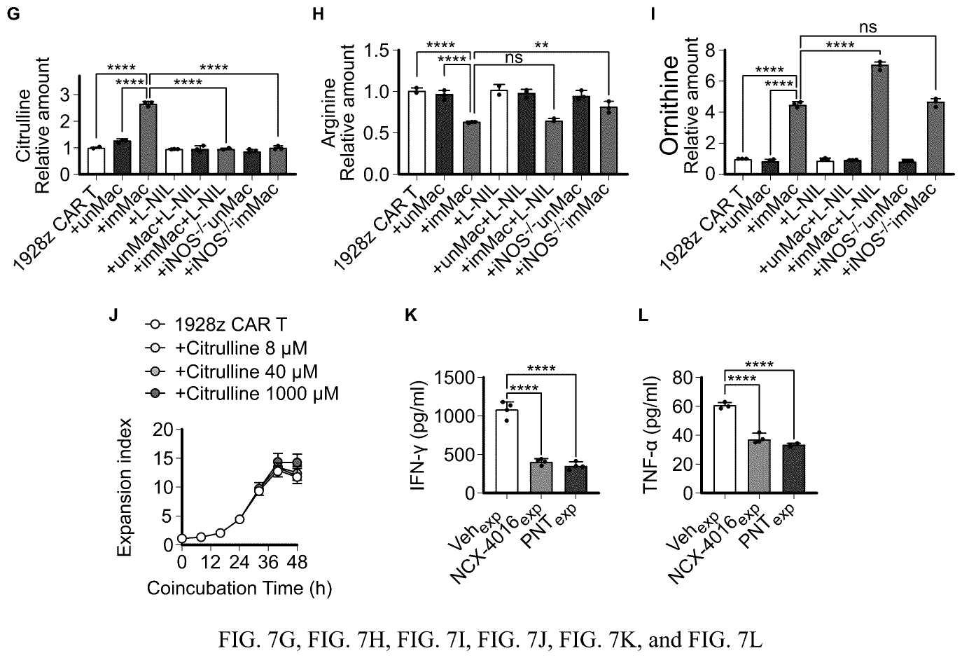

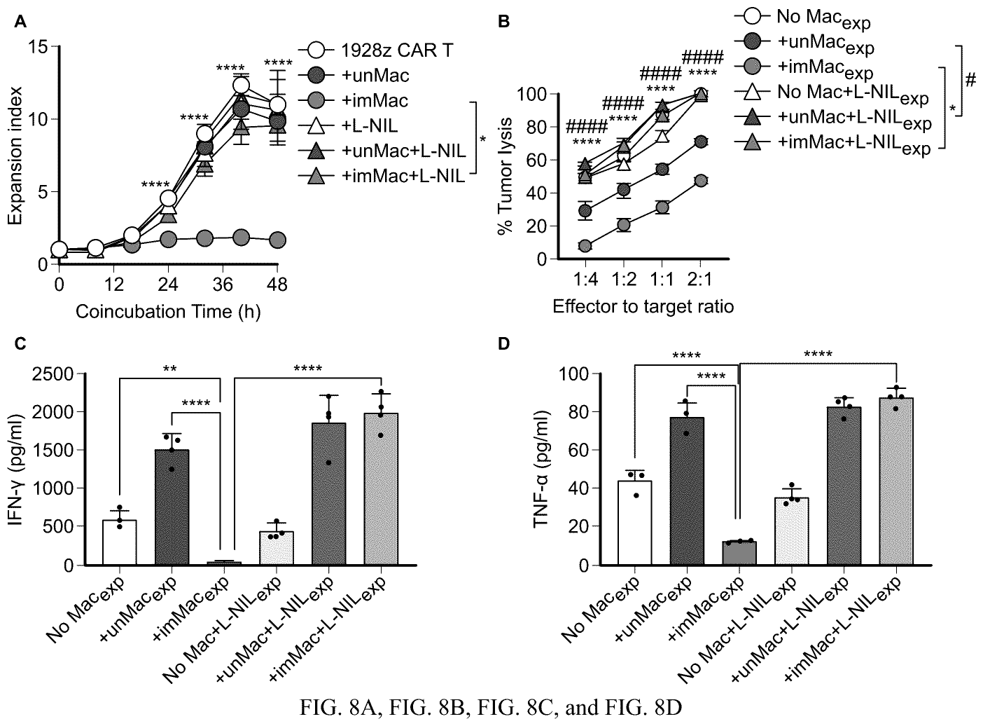

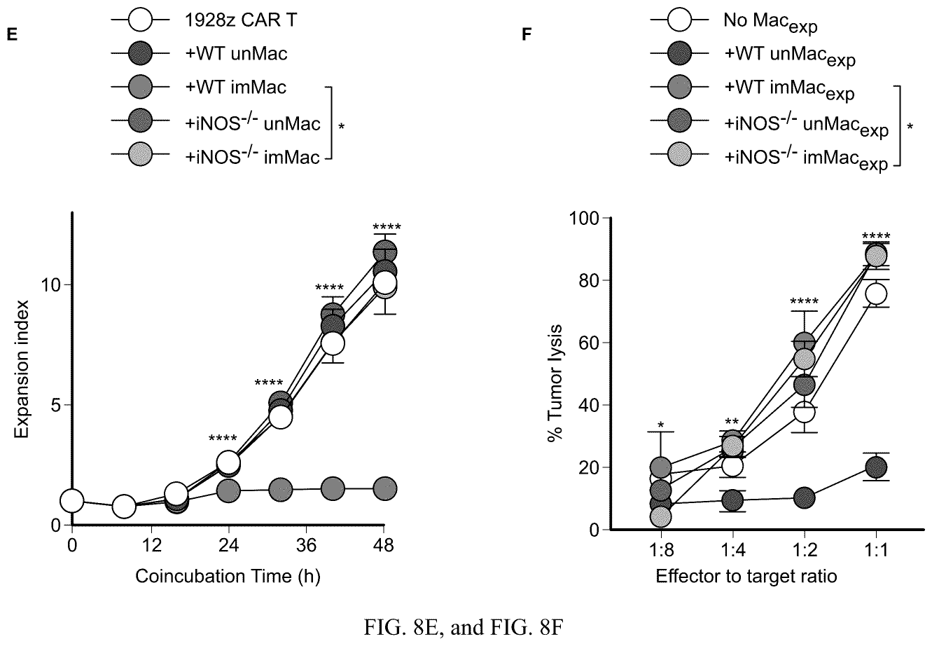

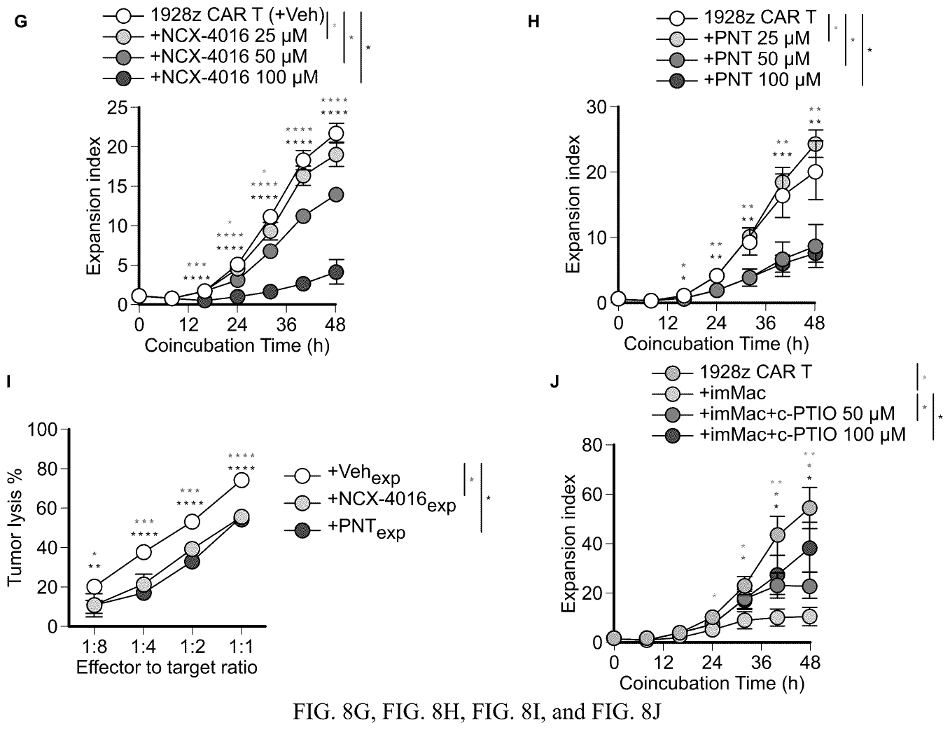

iNOS upregulation in imMac drives suppression of CAR T cell function: To investigate whether arginine metabolism by imMac contributes to the impairment of CAR T cell function, the study examined the effects of ARG-1 and iNOS inhibitors (FIG. 7A). Treatment with the ARG-1 inhibitor nor-NOHA⁴³ did not restore CAR T cell expansion in cocultures with imMac (FIG. 7B). The supraphysiological arginine concentration (1.15 mM) in culture media could diminish the effect of arginine depletion by ARG-1, thus the coculture was repeated at the physiological arginine concentration (150 μM). However, even in the context of physiological arginine levels, treatment with nor-NOHA failed to rescue CAR T cell expansion (FIG. 7C), suggesting ARG-1 by itself is not sufficient to inhibit CAR T cells. Next, the cocultures were treated with the iNOS inhibitor L-NIL, and a complete rescue of CAR T cell expansion in cocultures with imMac was observed (FIG. 8A). Furthermore, L-NIL treatment preserved the capacity of CAR T cells to kill tumor cells (FIG. 8B) and produce effector cytokines IFN-γ and TNF-α (FIG. 8C, FIG. 8D). Moreover, imMac developed from iNOS-deficient (iNOS^{-/-}) mice BMDMs did not inhibit CAR T cell expansion (FIG. 8E) or impair CAR T cell tumor killing capacity (FIG. 8F). Importantly, iNOS^{-/-} imMac expressed similar levels of ARG-1 and PD-L1 as wild-type (WT) imMac (FIG. 7D), indicating that these factors were not responsible for the suppression of CAR T cell function by imMac. Inhibition or genetic ablation of iNOS attenuated the production of NO (FIG. 7E, FIG. 7F) and citrulline (FIG. 7G) in the cocultures with imMac. The levels of arginine and ornithine remained comparable regardless of iNOS ablation or inhibition (FIG. 7H, FIG. 7I), ruling out their altered levels as the drivers of CAR T cell impairment. These findings collectively demonstrate that imMac suppresses CAR T cell function through the enzymatic activity of iNOS.

The study further investigated whether the iNOS products, citrulline and NO, were responsible for CAR T cell suppression by imMac. Exposure to high levels of citrulline did not impact the expansion of CAR T cells (FIG. 7J). NO reacts with superoxide to form peroxynitrite (PNT, ONOO⁻), which leads to protein oxidation, lipid peroxidation, and DNA damage. Treatment with the NO-donor NCX-4016 or PNT resulted in the inhibition of CAR T cell expansion (FIG. 8G, FIG. 8H) and diminished ability of CAR T cells to kill tumor cells (FIG. 8I) and secrete effector cytokines IFN-γ and TNF-α (FIG. 7K, FIG. 7L). Notably, treatment with NO-scavenger carboxyl-PTIO (c-PTIO) partially rescued the expansion of CAR T cells during cocultures with imMac (FIG. 8J). These data indicate that NO and PNT act as key mediators of iNOS-induced dysfunction of CAR T cells.

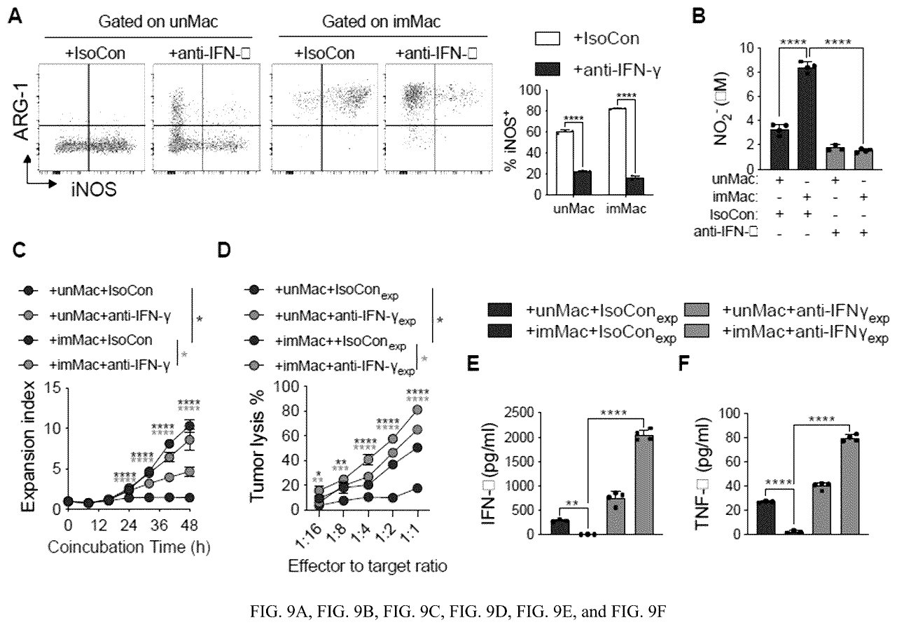

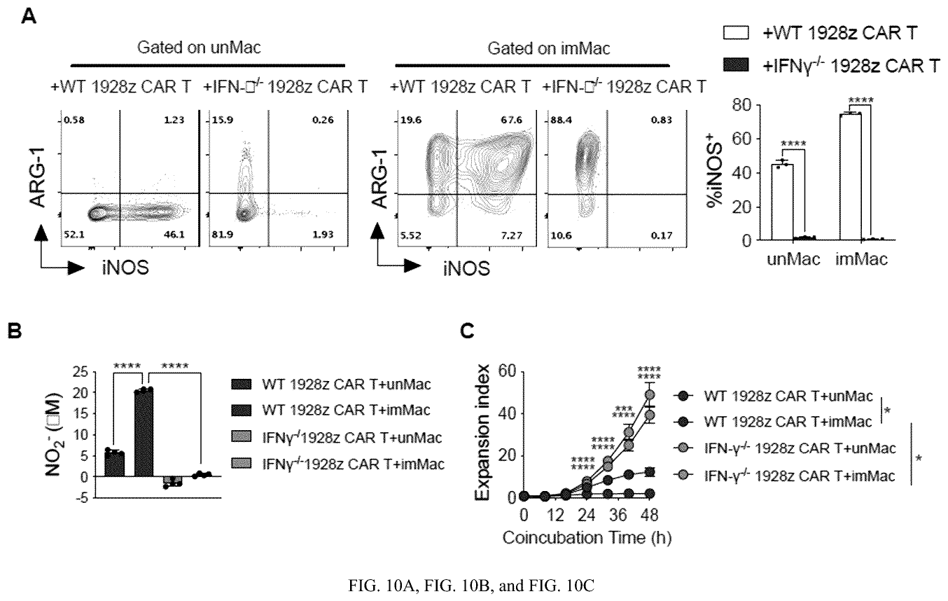

CAR T cell-derived IFN-γ induces iNOS in imMac: Secretion of cytokines, such as IFN-7 and TNF-α, by CAR T cells activates macrophages. A previous study reported that CAR T cell-derived IFN-γ upregulates iNOS in TAMs in a lung adenocarcinoma mouse model. It was found that neutralization of IFN-γ with blocking antibodies attenuated CAR T cell-triggered iNOS expression in unMac and imMac (FIG. 9A) and reduced the production of NO (FIG. 9B). In cocultures with imMac, treatment of anti-IFN-γ-enhanced CAR T cell expansion (FIG. 9C) and preserved the ability of CAR T cells to lyse tumor cells (FIG. 9D) and produce IFN-γ and TNF-α (FIG. 9E, FIG. 9F). Also, CAR T cells deficient in IFN-γ (IFN-γ^{-/-} CAR T cells) neither induced iNOS expression in unMac and imMac (FIG. 10A) nor induced NO production in the cocultures (FIG. 10B). Moreover, IFN-γ^{-/-} CAR T cells exhibited enhanced expansion during cocultures with imMac (FIG. 10C). These findings demonstrate that blocking IFN-γ in CAR T cells blunts counter-regulatory iNOS-driven inhibitory activity of imMac.

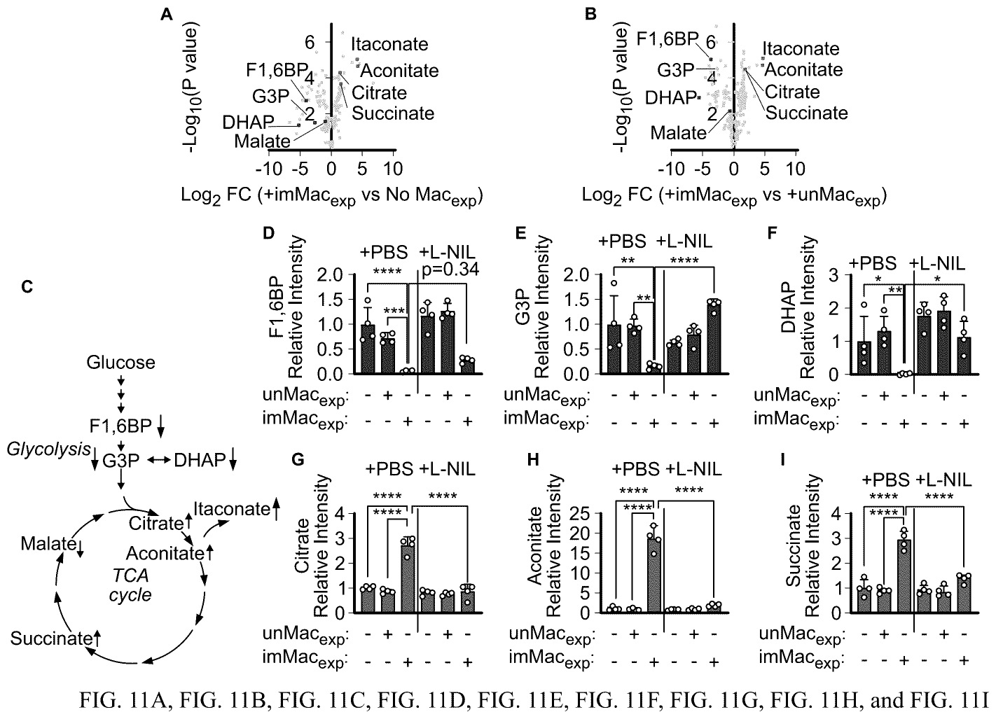

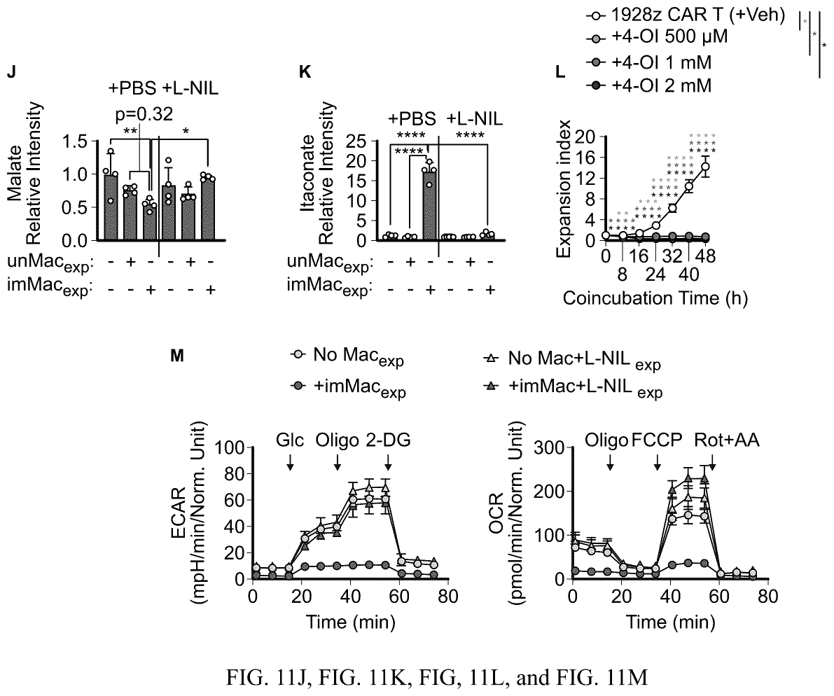

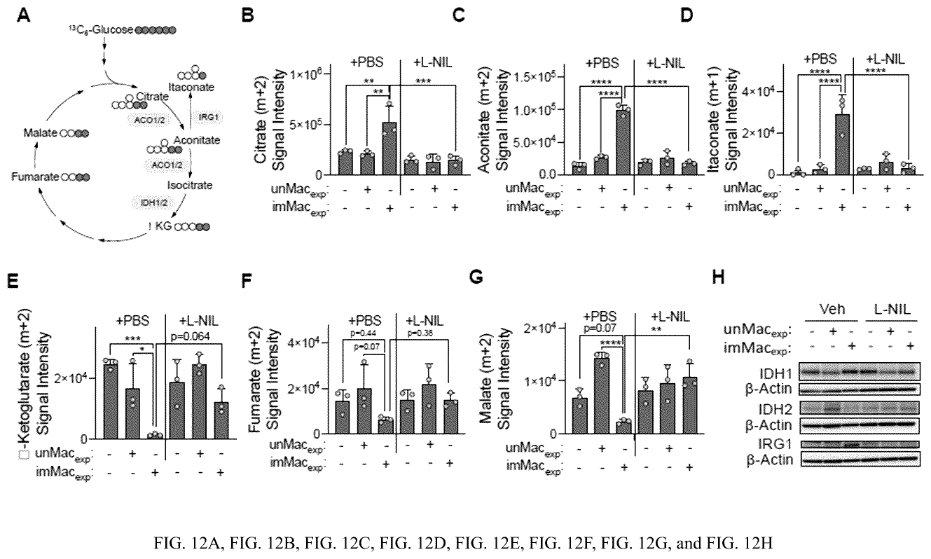

iNOS-expressing imMac induces CAR T cell metabolic dysregulation: Cellular metabolism plays a crucial role in supporting the rapid proliferation and effector function of T cells. To investigate the potential dysregulation of CAR T cell metabolism by imMac, global metabolomics analysis was conducted on CAR T cells from the coculture model. The study found significant alterations in glycolytic and TCA cycle intermediates in CAR T cells cocultured with imMac (FIG. 11A, FIG. 11B, FIG. 11C). Notably, the depletion of fructose 1,6-bisphosphate (F1,6BP), glyceraldehyde 3-phosphate (G3P), and dihydroxyacetone phosphate (DHAP) was found to be dependent on iNOS activity due to its reversal with L-NIL treatment (FIG. 11D, FIG. 11E, FIG. 11F). Moreover, there was an iNOS-dependent accumulation of citrate, aconitate, and succinate, along with a decrease in malate (FIG. 11G, FIG. 11H, FIG. 11I, FIG. 11J). The study also found a substantial accumulation of itaconate in CAR T cells cocultured with imMac (FIG. 11K). Itaconate is synthesized from aconitate via immune response gene 1 (IRG1), also known as aconitate decarboxylase (ACOD1), in tumor-associated myeloid cells and uptake of itaconate by CD8+ T cells has been shown to suppress their proliferation and cytolytic activity. Exposure to a cell-permeant form of itaconate, 4-octyl itaconate (4-OI), impaired CAR T cell expansion (FIG. 11L). Given the concurrent accumulation of itaconate with citrate and aconitate in CAR T cells cocultured with imMac, the study investigated whether CAR T cells can produce itaconate. Through ¹³C₆-glucose tracing on CAR T cells, the study identified iNOS-dependent accumulation of ¹³C-labeled citrate, aconitate, and itaconate, as well as a reduction of ¹³C₆-labeled α-ketoglutarate (αKG), fumarate, and malate in CAR T cells cocultured with imMac (FIG. 12A, FIG. 12B, FIG. 12C, FIG. 12D, FIG. 12E, FIG. 12F, FIG. 12G). Immunoblot analysis revealed the increased expression of IRG1 in CAR T cells cocultured with imMac, while the expression of isocitrate dehydrogenase 2 (IDH2) was decreased (FIG. 12H). These results indicate that imMac, via iNOS, induces metabolic vulnerability in CAR T cells by depleting glycolytic intermediates and rewiring the TCA cycle to divert aconitate towards itaconate production instead of αKG. Consistent with the altered metabolite profiles, extracellular flux analysis demonstrated that CAR T cells from cocultures with imMac exhibited attenuated glycolytic and oxidative metabolic activities, as evidenced by decreased extracellular acidification rate (ECAR) and oxygen consumption rate (OCR), which are proxies for glycolytic rate and mitochondrial oxidative phosphorylation, respectively (FIG. 11M). Importantly, L-NIL treatment preserved glycolytic and oxidative metabolic capacities of CAR T cells from cocultures with imMac. These findings demonstrate the broad disruption of CAR T cell metabolic programs by imMac, underscoring the importance of targeting iNOS to sustain the metabolic fitness of CAR T cells.

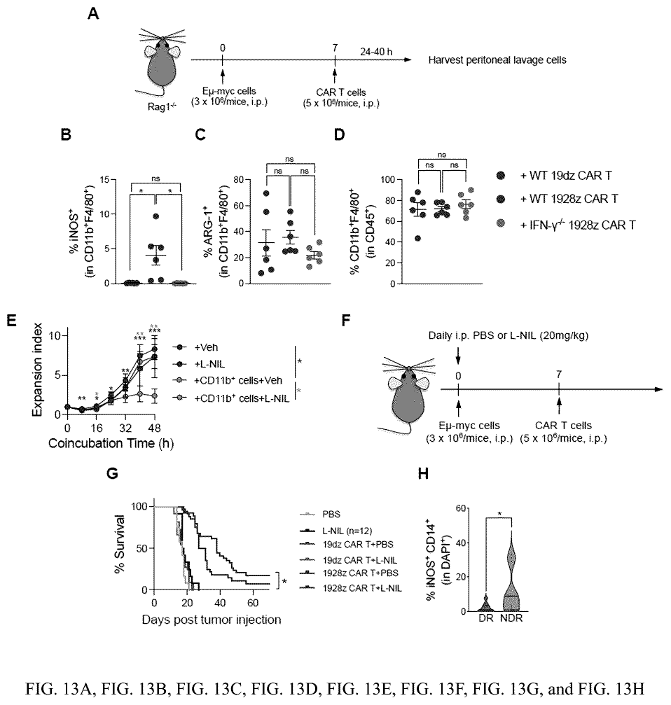

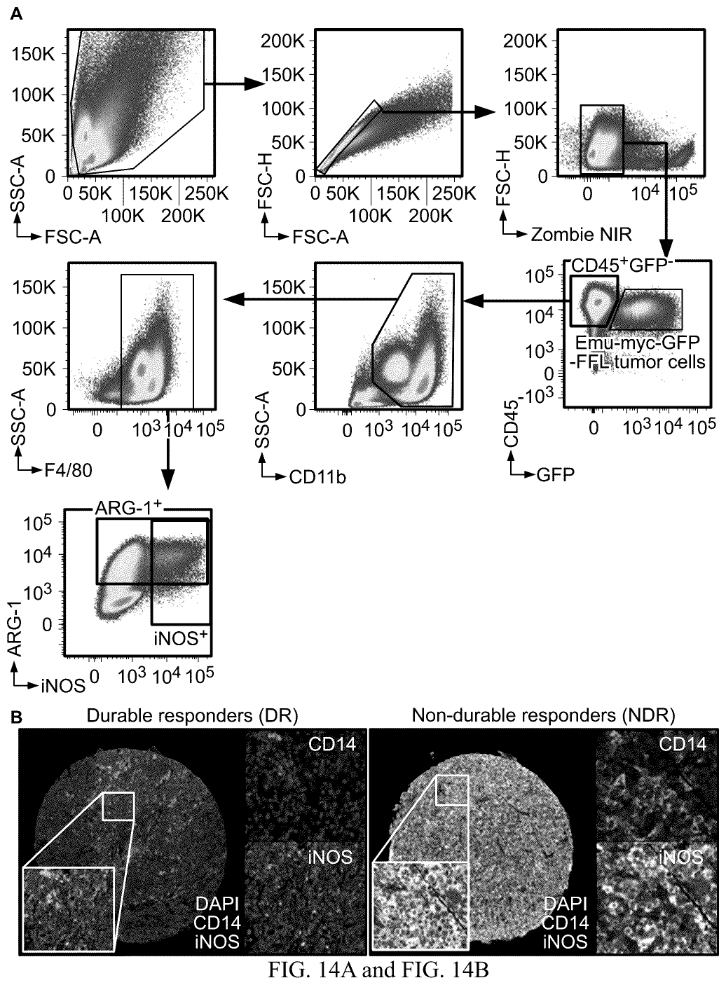

iNOS inhibition improves CAR T cell therapeutic efficacy: The study proceeded to investigate whether iNOS limits the effectiveness of CAR T cell therapy in vivo. To control the source of IFN-γ production, the study utilized Rag1^{-/-} mice, which lack endogenous T and B cells. Eμ-myc B cell tumors were established in the peritoneal cavity (FIG. 13A). CAR T cells carrying a truncated CD3ξ signaling domain (19dz) were used as non-functional controls (FIG. 4A). Intraperitoneal transfer of WT 1928z, IFN-γ^{-/-} 1928z, or WT 19dz CAR T cells was performed to facilitate direct interaction with macrophages at the tumor site. It was found that the frequency of iNOS+ macrophages was significantly elevated in WT 1928z CAR T cell-treated mice compared to mice treated with IFN-γ^{-/-} 1928z or WT 19dz CAR T cells (FIG. 13B, FIG. 14A). The frequencies of ARG1⁺ macrophages and total F4/80⁺CD11b⁺ macrophages were similar across all groups (FIG. 13C, FIG. 13D). Furthermore, CD11b⁺ myeloid cells from the peritoneal cavity of WT 1928z CAR T cell-treated tumor-bearing mice suppressed expansion of fresh antigen-naïve CAR T cells ex vivo in an iNOS-dependent manner (FIG. 13E). Next, the study assessed whether inhibition of iNOS could improve therapeutic efficacy of CAR T cells (FIG. 13F). Mice treated with a combination of 1928z CAR T cells and L-NIL exhibited significantly improved survival compared to mice treated with 1928z CAR T cells alone (FIG. 13G).

These results demonstrate that IFN-γ-producing CAR T cells stimulate iNOS in macrophages at the tumor site, and concurrent inhibition of iNOS enhances the therapeutic effectiveness of CAR T cells. While iNOS expression in macrophages has been attributed to induction of the CRS after CAR T cell treatment, it has not been noted to be associated with therapeutic resistance in patients. Therefore, to investigate whether iNOS expression correlates to clinical outcomes in patients receiving CD19-targeted CAR T cells, the study evaluated the expression of iNOS in CD14⁺ myeloid cells within pre-infusion patient tumors. Multiplex immunofluorescence analysis on tumor tissue microarrays (FIG. 2C) confirmed a higher proportion of iNOS⁺CD14⁺ cells in patients with NDR compared to patients with DR (FIG. 13H, FIG. 14B). These data provide further support that iNOS expression by myeloid cells within the TME contributes to non-durable responses to CAR T cell therapy in LBCL patients.

### DISCUSSION

The findings reported herein demonstrate that the upregulation of iNOS in imMac, provoked by IFN-γ secreted by CAR T cells, impairs various aspects of CAR T cell biology, including expansion, effector function, and metabolism, all of which can reduce the therapeutic efficacy of CAR T cells. iNOS is commonly referred to as a M1 macrophage-associated marker, which promotes anti-tumor effects. It was found that when imMac encounters CAR T cells, iNOS is co-expressed with M2 macrophage-associated markers, and has increased immunosuppressive potential. A high density of M2-like macrophages within the TME has been associated with poor prognosis in multiple cancer types owing to their tolerogenic properties. However, it is increasingly recognized that TAM phenotypes are complex and defy simple categorization into M1-M2 binary states. In line with these observations, increased iNOS in various cancers has been correlated with unfavorable prognoses, highlighting the tumor-promoting potential of iNOS. Notably, these data show that, despite the expression of iNOS in unMac, they do not exert suppressive effects on CAR T cells. This disparity might be attributed to the lower extent of iNOS upregulation and NO production in unMac compared to imMac. Moreover, under conditions of low arginine levels, iNOS catalyzes the production of superoxide, leading to the formation of PNT. This may, in part, account for the significant suppression of CAR T cells by iNOS⁺ ARG-1⁺ imMac compared to iNOS⁺ ARG-1⁻ unMac. The precise molecular mechanisms underlying this suppression remain to be elucidated. Further investigations into the transcriptional and translational regulation of CAR T cells by iNOS-expressing imMac are warranted. Nevertheless, the capacity of CAR T cells to induce iNOS in host TAMs emphasizes the need to develop CAR T cell treatment strategies that modulate iNOS in myeloid cells within the TME.

The abundance of iNOS⁺CD14⁺ myeloid cells is high within the pre-CAR T cell treatment TME of NDR patients. This finding supports the previous observation that patients with poor outcomes exhibit an inflammatory state, but this new data suggests that CAR T cells further intensify iNOS expression in TAMs. Therefore, future research characterizing iNOS expression in the TME both before and after CAR T cell infusion will be important for confirming these results. Pre-clinical and clinical data show that patients at high-risk for unfavorable outcomes can be identified by elevated pre-treatment baseline levels of serum inflammatory markers IL-6, c-reactive protein (CRP), and Ferritin. Interventional trials focusing on this patient subset can be developed to overcome suppressive macrophages within the TME and understand the relationship between TAM phenotype, IL-6, CRP, and Ferritin. The objective with these translational efforts is to increase the probability of achieving more frequent durable responses from CAR T cell therapy.

CAR T cell dysfunction induced by iNOS-expressing imMac involves repression of glycolytic and oxidative metabolic capacity. The metabolic profiles of CAR T cells have been shown to be crucial for their antitumor activity, persistence, and differentiation into memory T cells. Thus, the contribution of TME-associated factors in shaping CAR T cell metabolism might determine CAR T cell behavior and treatment outcomes. This study unveils a rewiring of the TCA cycle in CAR T cells, resulting in the accumulation of itaconate triggered by iNOS-expressing imMac. Notably, the expression of IRG1 and the production of itaconate were previously identified in macrophages and MDSCs, but not in T or natural killer (NK) cells, as immunosuppressive mediators. The mechanisms underlying the stabilization of IRG1 in CAR T cells and the extent to which endogenous production of itaconate contributes to CAR T cell dysfunction is an important future research direction. Understanding the regulation of CAR T cell metabolism within TME and developing strategies to sustain their metabolic fitness will enhance the efficacy and durability of CAR T cell therapy.

This work highlights the role of IFN-γ as an initiator of the iNOS-dependent inhibitory circuit between CAR T cells and imMac. Blocking IFN-γ in CAR T cells effectively eliminates the suppressive effects mediated by imMac. The role of IFN-γ in the TME is paradoxical, as it can promote tumor cell apoptosis but can also limit antitumor immunity by upregulation of inhibitory molecules, such as PD-L1, PD-L2, indoleamine 2,3-dioxygenase 1 (IDO), FAS, and FAS ligand (FASL). Similarly, CAR T cell-derived IFN-γ has been shown to activate host antitumor immunity and sustain CAR T cell cytotoxicity but can also induce macrophage production of cytokines and chemokines associated with CRS. Neutralizing IFN-γ mitigated CAR T therapy-associated toxicity without compromising the antitumor efficacy of CAR T cells against lymphoma and leukemia. Moreover, the tumor cytotoxic effects of CAR T cell-derived IFN-γ are dependent on the intrinsic sensitivity of cancer cells to IFN-γ signaling. Therefore, the impact of IFN-γ on the TME and tumor progression is determined by various factors, including immune cell composition, cellular phenotypes, and tumor genetics. Further investigations are needed to determine the extent to which targeting IFN-γ can enhance CAR T therapy in a tumor type-specific manner.

### Example 2: IRG Blockade to Armor CAR T Cells Against Myeloid Dysfunction

Macrophage drives CAR T cell failure via endogenous itaconate accumulation: A study was conducted which determined that CAR T cells are rendered dysfunctional through metabolic dysregulation of the citric acid cycle. A key enzyme in this pathway the results in the metabolic dysfunction is IRG1. Therefore, blockade of IRG1 by genetic deletion, gene suppression, or other means will render the CAR T cells resistant to metabolic dysfunction.

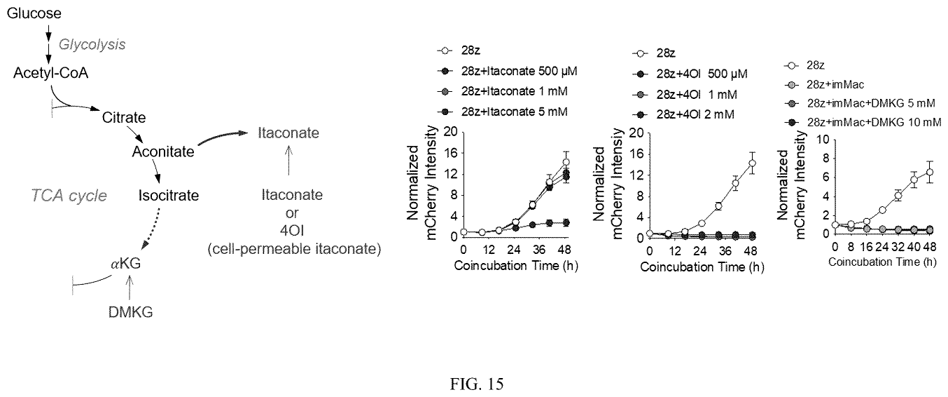

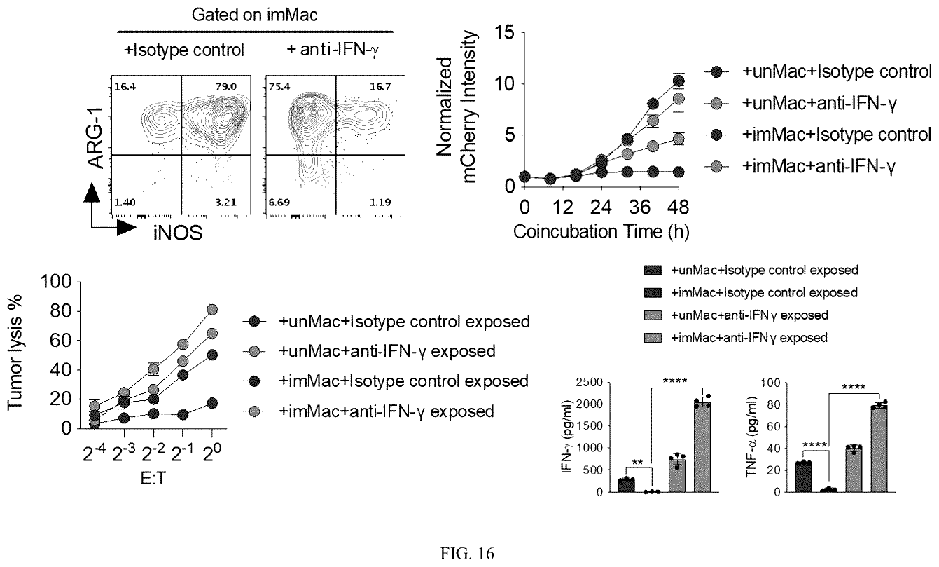

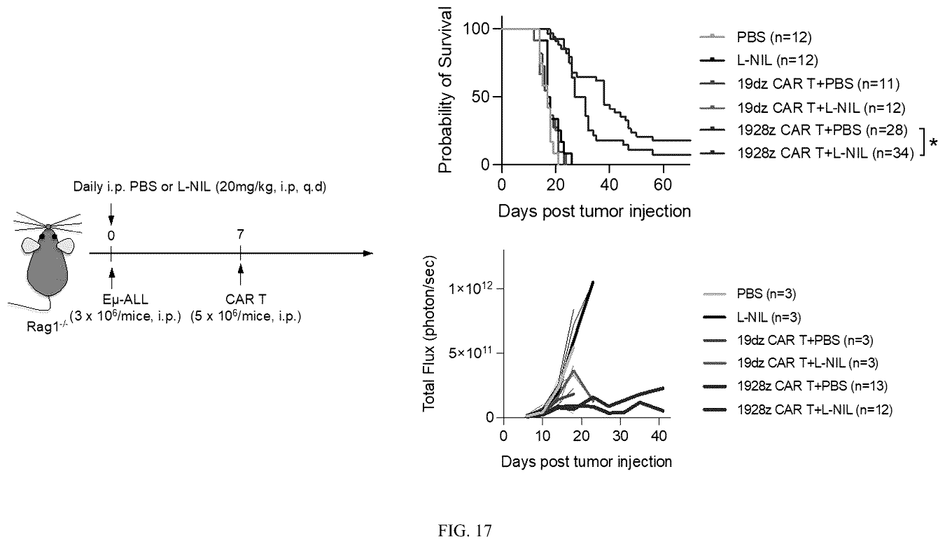

FIG. 2A and FIG. 2B show the presence of immunoregulatory macrophages in pre-treatment TME associated with axi-cell failure in LBCL. FIG. 3A, FIG. 3B, FIG. 3C, FIG. 3D, FIG. 3E, FIG. 3F, FIG. 3G, FIG. 3H, FIG. 3I, and FIG. 3J show that exposure of immunoregulatory macrophage to CAR T cells provokes CAR T cell dysfunction. FIG. 5A, FIG. 5B, FIG. 5C, FIG. 5D, FIG. 5E, FIG. 5F, FIG. 5G, and FIG. 5H show that CAR T cells promote immunoregulatory metabolic axis in imMac via iNOS upregulation. FIG. 8A, FIG. 8B, FIG. 8C, FIG. 8D, FIG. 8E, FIG. 8F, FIG. 8G, FIG. 8H, FIG. 8I, and FIG. 8J show that CAR T-induced iNOS upregulation in imMac drives suppression of CAR T cell function, which was demonstrated via iNOS inhibitor L-NIL (FIG. 8A, FIG. 8B, FIG. 8C, FIG. 8D) and via iNOS KO macrophage (FIG. 8E, FIG. 8F). FIG. 12 show that iNOS in imMac disrupts TCA cycle progression in CAR T cells via accumulation of citrate (FIG. 12B), aconitate (FIG. 12C), and itaconate (FIG. 12D) and depletion of α-ketoglutarate (FIG. 12E), and via decreased expression of ACO1 and IDH2 and increased expression of IRG1, responsible for TCA cycle break in CAR T cells (FIG. 12H). Treatment of itaconate or 4OI (cell-permeable itaconate) reduced CAR T cell expansion, while supplementation of DMKG (cell-permeable α-ketoglutarate) failed to rescue CAR T cell expansion. Endogenous itaconate production and accumulation may be driving CAR T failure (FIG. 15). FIG. 16 shows that CAR T cell-derived IFN-γ spurs upregulation of iNOS in imMac. FIG. 17 shows that iNOS inhibition improves CAR T cell-mediated tumor control and survival.

Accordingly, this study has determined that CAR T cells are rendered dysfunctional through metabolic dysregulation of the citric acid cycle. A key enzyme in this pathway the results in the metabolic dysfunction is IRG1. Therefore blockade of IRG1 by genetic deletion, gene suppression, or other means will render the CAR T cells resistant to metabolic dysfunction

The following patents, applications and publications as listed below and throughout this document are hereby incorporated by reference in their entirety herein.

### REFERENCE LIST

Adams, J. M. et al. The c-myc oncogene driven by immunoglobulin enhancers induces lymphoid malignancy in transgenic mice. Nature 318, 533-538 (1985).
Alizadeh, D. et al. IFNgamma Is Critical for CAR T Cell-Mediated Myeloid Activation and Induction of Endogenous Immunity. Cancer Discov 11, 2248-2265 (2021).
Bailey, S. R. et al. Blockade or Deletion of IFNgamma Reduces Macrophage Activation without Compromising CAR T-cell Function in Hematologic Malignancies. Blood Cancer Discov 3, 136-153 (2022).
Bantug, G. R., Galluzzi, L., Kroemer, G. & Hess, C. The spectrum of T cell metabolism in health and disease. Nat Rev Immunol 18, 19-34 (2018).
Boucher, J. C. et al. CD28 Costimulatory Domain-Targeted Mutations Enhance Chimeric Antigen Receptor T-cell Function. Cancer Immunol Res 9, 62-74 (2021).
Boulch, M. et al. A cross-talk between CAR T cell subsets and the tumor microenvironment is essential for sustained cytotoxic activity. Sci Immunol 6 (2021).
Boulch, M. et al. Tumor-intrinsic sensitivity to the pro-apoptotic effects of IFN-gamma is a major determinant of CD4(+) CAR T-cell antitumor activity. Nat Cancer (2023).
Bronte, V. & Zanovello, P. Regulation of immune responses by L-arginine metabolism. Nat Rev Immunol 5, 641-654 (2005).
Bruni, D., Angell, H. K. & Galon, J. The immune contexture and Immunoscore in cancer prognosis andtherapeutic efficacy. Nat Rev Cancer 20, 662-680 (2020).
Cappell, K. M. & Kochenderfer, J. N. Long-term outcomes following CAR T cell therapy: what we know so far. Nat Rev Clin Oncol, 1-13 (2023).
Cassetta, L. & Pollard, J. W. A timeline of tumour-associated macrophage biology. Nat Rev Cancer 23, 238-257 (2023).
Chang, C. H. et al. Metabolic Competition in the Tumor Microenvironment Is a Driver of Cancer Progression. Cell 162, 1229-1241 (2015).
Chen, C. N., Hsieh, F. J., Cheng, Y. M., Chang, K. J. & Lee, P. H. Expression of inducible nitric oxide synthase and cyclooxygenase-2 in angiogenesis and clinical outcome of human gastric cancer. J Surg Oncol 94, 226-233 (2006).
Chen, Y. J. et al. Targeting IRG1 reverses the immunosuppressive function of tumor-associated macrophages and enhances cancer immunotherapy. Sci Adv 9, eadg0654 (2023).
Christofides, A. et al. The complex role of tumor-infiltrating macrophages. Nat Immunol 23, 1148-1156(2022).
Davila, M. L., Kloss, C. C., Gunset, G. & Sadelain, M. CD19 CAR-targeted T cells induce long-term remission and B Cell Aplasia in an immunocompetent mouse model of B cell acute lymphoblastic leukemia. PLoS One 8, e61338 (2013).
Dean, E. A. et al. High metabolic tumor volume is associated with decreased efficacy of axicabtagene ciloleucel in large B-cell lymphoma. Blood Adv 4, 3268-3276 (2020).
Deng, Q. et al. Characteristics of anti-CD19 CAR T cell infusion products associated with efficacy and toxicity in patients with large B cell lymphomas. Nat Med 26, 1878-1887 (2020).
Eyler, C. E. et al. Glioma stem cell proliferation and tumor growth are promoted by nitric oxide synthase-2. Cell 146, 53-66 (2011).
Faramand, R. et al. Tumor Microenvironment Composition and Severe Cytokine Release Syndrome (CRS) Influence Toxicity in Patients with Large B-Cell Lymphoma Treated with Axicabtagene Ciloleucel. Clin Cancer Res 26, 4823-4831 (2020).
Fraietta, J. A. et al. Determinants of response and resistance to CD19 chimeric antigen receptor (CAR) T cell therapy of chronic lymphocytic leukemia. Nat Med 24, 563-571 (2018).
Fridman, W. H., Zitvogel, L., Sautes-Fridman, C. & Kroemer, G. The immune contexture in cancer prognosis and treatment. Nat Rev Clin Oncol 14, 717-734 (2017).
Gemta, L. F. et al. Impaired enolase 1 glycolytic activity restrains effector functions of tumor-infiltrating CD8(+) T cells. Sci Immunol 4 (2019).
Giavridis, T. et al. CAR T cell-induced cytokine release syndrome is mediated by macrophages and abated by IL-1 blockade. Nat Med 24, 731-738 (2018).
Glynn, S. A. et al. Increased NOS2 predicts poor survival in estrogen receptor-negative breast cancer patients. J Clin Invest 120, 3843-3854 (2010).
Gocher, A. M., Workman, C. J. & Vignali, D. A. A. Interferon-gamma: teammate or opponent in the tumour microenvironment? Nat Rev Immunol 22, 158-172 (2022).
Granados-Principal, S. et al. Inhibition of iNOS as a novel effective targeted therapy against triple-negative breast cancer. Breast Cancer Res 17, 25 (2015).
Harris, A. W. et al. The E mu-myc transgenic mouse. A model for high-incidence spontaneous lymphoma and leukemia of early B cells. J Exp Med 167, 353-371 (1988).
Harris, I. S. & DeNicola, G. M. The Complex Interplay between Antioxidants and ROS in Cancer. Trends Cell Biol 30, 440-451 (2020).
Ho, P. C. et al. Phosphoenolpyruvate Is a Metabolic Checkpoint of Anti-tumor T Cell Responses. Cell 162, 1217-1228 (2015).
Jackson, Z. et al. Sequential Single-Cell Transcriptional and Protein Marker Profiling Reveals TIGIT as a Marker of CD19 CAR-T Cell Dysfunction in Patients with Non-Hodgkin Lymphoma. Cancer Discov 12,1886-1903 (2022).
Jain, M. D. et al. Tumor interferon signaling and suppressive myeloid cells are associated with CAR T-cell failure in large B-cell lymphoma. Blood 137, 2621-2633 (2021).
Jain, M. D. et al. Whole-genome sequencing reveals complex genomic features underlying anti-CD19 CART-cell treatment failures in lymphoma. Blood 140, 491-503 (2022).
Kang, Y. P. et al. Non-canonical Glutamate-Cysteine Ligase Activity Protects against Ferroptosis. Cell Metab 33, 174-189 e177 (2021).
Kawalekar, O. U. et al. Distinct Signaling of Coreceptors Regulates Specific Metabolism Pathways and Impacts Memory Development in CAR T Cells. Immunity 44, 380-390 (2016).
Kloosterman, D. J. & Akkari, L. Macrophages at the interface of the co-evolving cancer ecosystem. Cell 186, 1627-1651 (2023).
Larson, R. C. et al. CAR T cell killing requires the IFNgammaR pathway in solid but not liquid tumours. Nature 604, 563-570 (2022).
Liu, Y. et al. Gasdermin E-mediated target cell pyroptosis by CAR T cells triggers cytokine release syndrome. Sci Immunol 5 (2020).
Locke, F. L. et al. Axicabtagene Ciloleucel as Second-Line Therapy for Large B-Cell Lymphoma. N Engl J Med 386, 640-654 (2022).
Locke, F. L. et al. Long-term safety and activity of axicabtagene ciloleucel in refractory large B-cell lymphoma (ZUMA-1): a single-arm, multicentre, phase 1-2 trial. Lancet Oncol 20, 31-42 (2019).
Locke, F. L. et al. Phase 1 Results of ZUMA-1: A Multicenter Study of KTE-C19 Anti-CD19 CAR T Cell Therapy in Refractory Aggressive Lymphoma. Mol Ther 25, 285-295 (2017).
Locke, F. L. et al. Tumor burden, inflammation, and product attributes determine outcomes of axicabtagene ciloleucel in large B-cell lymphoma. Blood Adv 4, 4898-4911 (2020).
Majzner, R. G. & Mackall, C. L. Clinical lessons learned from the first leg of the CAR T cell journey. Nat Med 25, 1341-1355 (2019).
Majzner, R. G. et al. Tuning the Antigen Density Requirement for CAR T-cell Activity. Cancer Discov 10, 702-723 (2020).
Manni, S. et al. Neutralizing IFNgamma improves safety without compromising efficacy of CAR-T cell therapy in B-cell malignancies. Nat Commun 14, 3423 (2023).
Mantovani, A., Allavena, P., Marchesi, F. & Garlanda, C. Macrophages as tools and targets in cancer therapy. Nat Rev Drug Discov 21, 799-820 (2022).
Martinez, F. O., Gordon, S., Locati, M. & Mantovani, A. Transcriptional profiling of the human monocyte-to-macrophage differentiation and polarization: new molecules and patterns of gene expression. J Immunol 177, 7303-7311 (2006).
Maude, S. L. et al. Chimeric antigen receptor T cells for sustained remissions in leukemia. N Engl J Med 371, 1507-1517 (2014).
Maude, S. L. et al. Tisagenlecleucel in Children and Young Adults with B-Cell Lymphoblastic Leukemia. N Engl J Med 378, 439-448 (2018).
Millard, P. et al. IsoCor: isotope correction for high-resolution MS labeling experiments. Bioinformatics 35, 4484-4487 (2019).
Mills, E. L. et al. Itaconate is an anti-inflammatory metabolite that activates Nrf2 via alkylation of KEAP1. Nature 556, 113-117 (2018).
Moore, W. M. et al. L-N6-(1-iminoethyl)lysine: a selective inhibitor of inducible nitric oxide synthase. J Med Chem 37, 3886-3888 (1994).
Morris, E. C., Neelapu, S. S., Giavridis, T. & Sadelain, M. Cytokine release syndrome and associated neurotoxicity in cancer immunotherapy. Nat Rev Immunol 22, 85-96 (2022).
Munn, D. H. et al. Inhibition of T cell proliferation by macrophage tryptophan catabolism. J Exp Med 189,1363-1372 (1999).
Nathan, C. & Cunningham-Bussel, A. Beyond oxidative stress: an immunologist's guide to reactive oxygen species. Nat Rev Immunol 13, 349-361 (2013).
Neelapu, S. S. et al. Axicabtagene Ciloleucel CAR T-Cell Therapy in Refractory Large B-Cell Lymphoma. N Engl J Med 377, 2531-2544 (2017).
Newman, A. M. et al. Determining cell type abundance and expression from bulk tissues with digital cytometry. Nat Biotechnol 37, 773-782 (2019).
Newman, A. M. et al. Robust enumeration of cell subsets from tissue expression profiles. Nat Methods 12, 453-457 (2015).
Norelli, M. et al. Monocyte-derived IL-1 and IL-6 are differentially required for cytokine-release syndrome and neurotoxicity due to CAR T cells. Nat Med 24, 739-748 (2018).
Park, J. H. et al. Long-Term Follow-up of CD19 CAR Therapy in Acute Lymphoblastic Leukemia. N Engl J Med 378, 449-459 (2018).
Plaks, V. et al. CD19 target evasion as a mechanism of relapse in large B-cell lymphoma treated with axicabtagene ciloleucel. Blood 138, 1081-1085 (2021).
Reina-Campos, M., Scharping, N. E. & Goldrath, A. W. CD8(+) T cell metabolism in infection and cancer. Nat Rev Immunol 21, 718-738 (2021).
Rodriguez, P. C. & Ochoa, A. C. Arginine regulation by myeloid derived suppressor cells and tolerance in cancer: mechanisms and therapeutic perspectives. Immunol Rev 222, 180-191 (2008).
Rodriguez, P. C. et al. Arginase I production in the tumor microenvironment by mature myeloid cells inhibits T-cell receptor expression and antigen-specific T-cell responses. Cancer Res 64, 5839-5849 (2004).
Roselli, E., Faramand, R. & Davila, M. L. Insight into next-generation CAR therapeutics: designing CAR T cells to improve clinical outcomes. J Clin Invest 131 (2021).
Scharping, N. E. et al. The Tumor Microenvironment Represses T Cell Mitochondrial Biogenesis to Drive Intratumoral T Cell Metabolic Insufficiency and Dysfunction. Immunity 45, 374-388 (2016).
Scholler, N. et al. Tumor immune contexture is a determinant of anti-CD19 CAR T cell efficacy in large Bcell lymphoma. Nat Med 28, 1872-1882 (2022).
Schuster, S. J. et al. Tisagenlecleucel in Adult Relapsed or Refractory Diffuse Large B-Cell Lymphoma. N Engl J Med 380, 45-56 (2019).
Scott, D. W. & Gascoyne, R. D. The tumour microenvironment in B cell lymphomas. Nat Rev Cancer 14,517-534 (2014).
Shouval, R. et al. Impact of TP53 Genomic Alterations in Large B-Cell Lymphoma Treated with CD19-Chimeric Antigen Receptor T-Cell Therapy. J Clin Oncol 40, 369-381 (2022).
Singhal, S. et al. Human tumor-associated monocytes/macrophages and their regulation of T cell responses in early-stage lung cancer. Sci Transl Med 11 (2019).
Srivastava, S. et al. Immunogenic Chemotherapy Enhances Recruitment of CAR-T Cells to Lung Tumors and Improves Antitumor Efficacy when Combined with Checkpoint Blockade. Cancer Cell 39, 193-208 e110 (2021).
Steen, C. B. et al. The landscape of tumor cell states and ecosystems in diffuse large B cell lymphoma. Cancer Cell 39, 1422-1437 e1410 (2021).
Tenu, J. P. et al. Effects of the new arginase inhibitor N(omega)-hydroxy-nor-L-arginine on NO synthase activity in murine macrophages. Nitric Oxide 3, 427-438 (1999).
Thommen, D. S. & Schumacher, T. N. T Cell Dysfunction in Cancer. Cancer Cell 33, 547-562 (2018).
Yu, Y. R. et al. Disturbed mitochondrial dynamics in CD8(+) TILs reinforce T cell exhaustion. Nat Immunol 21, 1540-1551 (2020).
Zhao, H. et al. Myeloid-derived itaconate suppresses cytotoxic CD8(+) T cells and promotes tumour 700 growth. Nat Metab 4, 1660-1673 (2022)

1. A method of treating a cancer or an autoimmune disease in a subject comprising administering to the subject an adoptive immune cell immunotherapy and an agent that blocks dysregulation of the citric acid cycle.

2. The method of claim 1, wherein the agent that blocks dysregulation of the citric acid cycle is an agent that inhibits inducible nitric oxide synthase (iNOS) and/or an agent that inhibits immune responsive gene 1 (IRG1).

3. The method of claim 1, wherein the adoptive immune cell immunotherapy comprises the administration of chimeric antigen receptor (CAR) T cells, CAR natural killer (NK) cells, tumor infiltrating lymphocytes (TILs).

4. The method of claim 1, wherein the immune cells in the adoptive immune cell immunotherapy are CD8+ T cells, CD4+ T cells, NK cells, or NK T cells.

5. The method of claim 1, wherein the iNOS inhibitor is an antibody, antibody fragment, small molecule, siRNA, or oligonucleotide that disrupts iNOS transcription, expression, or binding.

6. The method of claim 5, wherein the iNOS inhibitor comprises N⁶-(1-Iminoethyl)-lysine, hydrochloride(L-NIL).

7. The method of claim 1, wherein the IRG1 inhibitor is an antibody, antibody fragment, small molecule, siRNA, or oligonucleotide that disrupts IRG1 transcription, expression, or binding.

8. A method of treating a cancer or an autoimmune disease in a subject comprising administering to the subject an adoptive immune cell immunotherapy wherein the immune cell has been modified to disrupt expression of immune responsive gene 1 (IRG1).

9. The method of claim 8, wherein the adoptive immune cell immunotherapy comprises the administration of chimeric antigen receptor (CAR) T cells, CAR natural killer (NK) cells, tumor infiltrating lymphocytes (TILs).

10. The method of claim 8, wherein the immune cells in the adoptive immune cell immunotherapy are CD8+ T cells, CD4+ T cells, NK cells, or NK T cells.

11. The method of claim 8, wherein the expression of IRG1 is disrupted by a clustered regularly interspaced short palindromic repeat (CRISPR)/CRISPR-associated (Cas)integration systems that targets iNOS or IRG1.

12. The method of claim 11, wherein the CRISPR/Cas integration system comprises a Class 2 CRISPR/Cas integration system.

13. The method of claim 12, wherein the class 2 CRISPR/Cas system comprises a CRISPR/Cas9 integration system.

14. The method of claim 8, wherein the immune cell is modified ex vivo.

15. A modified chimeric antigen receptor (CAR) immune cell or tumor infiltrating lymphocyte (TIL) comprising a disrupted immune responsive gene 1 (IRG1) gene.

16. The CAR immune cell of claim 15, wherein the CAR immune cell or TIL is a CD8+ T cell, CD4+ T cell, NK cell, or NK T cell.

17. A method of treating a cancer disease, or an autoimmune disease in a subject comprising administering to the subject the CAR immune cell or TIL of claim 15.

18. A method of rescuing non-durable responses (NDR) to an adoptive immune cell immunotherapy for a cancer or an autoimmune disease in a subject comprising
a) obtaining previously adoptively transferred immune cells from the subject,
b) measuring the expression level of immune responsive gene 1 (IRG1) in the transferred immune cell; and
c) administering to the subject an agent that inhibits IRG1 or inducible nitric oxide synthase (iNOS) or administering to the subject chimeric antigen receptor (CAR) immune cell comprising a IRG1 gene when the expression level of IRG1 in the adoptively transferred immune cell has increased relative to a control.

19. A method of rescuing non-durable responses (NDR) to an adoptive immune cell immunotherapy for a cancer or an autoimmune disease in a subject comprising
a) obtaining macrophage in the tumor microenvironment (TME) from the subject,
b) measuring the expression level of iNOS in the macrophage; and
c) administering to the subject an agent that inhibits immune responsive gene 1 (IRG1) or iNOS or administering to the subject chimeric antigen receptor (CAR) immune cell comprising a disrupted IRG1 gene when the expression level of iNOS in the TME macrophage has increased relative to a control.
